(19) United States
(12) Patent Application Publication
(10) Pub. No.: US 2024/0342350 A1
(43) Pub. Date: Oct. 17, 2024

(54) SYSTEMS AND METHODS FOR DIALYSIS FLUID PREPARATION IN BATCH DISPOSABLE

(71) Applicants: BAXTER INTERNATIONAL INC., Deerfield, IL (US); BAXTER HEALTHCARE SA, Glattpark (Opfikon) (CH)

(72) Inventors: Christian Antero VARTIA, Veberöd (SE); Olof JANSSON, Vellinge (SE); Sture HOBRO, Lund (SE)

(21) Appl. No.: 18/579,736

(22) PCT Filed: Jun. 13, 2022
(86) PCT No.: PCT/US2022/033243

§ 371 Date: Jan. 16, 2024

Related U.S. Application Data
(60) Provisional application No. 63/222,643, filed on Jul. 16, 2021.

(51) Int. Cl. A61M 1/14; A61M 1/28

(52) U.S. Cl. CPC A61M 1/159; A61M 1/153; A61M 1/154; A61M 1/155; A61M 1/1566; A61M 1/287; A61M 2205/3317; A61M 2205/3393

(57) ABSTRACT

A peritoneal dialysis system includes a water purifier; a disposable set including a water line in fluid communication with the water purifier, a drain line for draining from the disposable set, and a disposable container including at least one chamber, the disposable container including at least one concentrate in one of the at least one chamber, and the disposable container positioned and arranged to hold a dialysis fluid prepared by mixing water from the water purifier and the at least one concentrate; and a control unit in communication with at least one sensor, for detecting a first property of water from the water purifier and a second property of the dialysis fluid.

### PRIORITY CLAIM

The present application claims priority to and the benefit of U.S. Provisional Application No. 63/222,643, filed Jul. 16, 2021, the entire contents of which are incorporated herein by reference and relied upon.

### BACKGROUND

The present invention relates to the field of fluid compounding for preparing fluids particularly for the treatment of renal insufficiency. More specifically, it relates to a batch disposable system and apparatus for preparing dialysis fluid for the treatment of renal insufficiency. The systems and methods disclosed herein are configured for compounding finished fluids from two or more constituent fluids, liquid concentrates or dry concentrates for use as a kidney dialyzing fluid.

In particular, the invention may be used for preparing fluids for peritoneal dialysis, particularly for preparing fluids on-site (e.g. at patient's home).

The kidneys fulfil many functions, including the removal of water, the excretion of catabolites (or waste from the metabolism, for example urea and creatinine), the regulation of the concentration of the electrolytes in the blood (e.g. sodium, potassium, magnesium, calcium, bicarbonate, phosphate, chloride) and the regulation of the acid/base equilibrium within the body, which is obtained in particular by the removal of weak acids (phosphates, monosodium acids) and by the production of ammonium salts.

In individuals who have lost the use of their kidneys, since the kidney excretion and regulation mechanisms no longer work properly, the body accumulates water and waste from the metabolism and exhibits an excess of electrolytes. The pH of an individual's blood plasma shifts downwards, below 7.35 (the blood pH normally varies within narrow limits of between 7.35 and 7.45), due to their kidneys' failure to regenerate and replenish the blood with bicarbonate. Reduced bicarbonate levels lead to reduced pH.

In the treatment of patients suffering acute or chronic renal insufficiency, dialysis therapy is employed. The two general categories of dialysis therapy are hemodialysis and peritoneal dialysis.

In hemodialysis, the patient's blood is cleansed by passage through an artificial kidney in an extracorporeal membrane system.

The blood treatment involves extracorporeal circulation through an exchanger having a semipermeable membrane (dialyzer) in which the patient's blood is circulated on one side of the membrane while a dialysis liquid, comprising the main electrolytes of the blood in concentrations close to those in the blood of a healthy subject, is circulated on the other side.

Further, a pressure difference is created between the two compartments of the dialyzer, which are delimited by the semipermeable membrane, so that a fraction of the plasma fluid passes by ultrafiltration through the membrane into the compartment containing the dialysis liquid.

In peritoneal dialysis, dialyzing fluid is infused into the patient's peritoneal cavity. This cavity is lined by the peritoneal membrane which is highly vascularized. The metabolites are removed from the patient's blood by diffusion across the peritoneal membrane into the dialyzing fluid. Excess fluid, i.e., water is also removed by osmosis induced by a hypertonic dialyzing fluid.

When an aqueous solution is instilled into the peritoneal cavity, the solute composition equilibrates with that of plasma water by passive diffusion along electrochemical concentration gradients. In addition, the flux of fluid across the peritoneum in response to an osmotic agent moves solutes in the absence of a concentration gradient, leading to the concept that solute transport occurs partly by convection or ‘solvent drag’. Removal of excess fluid is achieved by adding to the solution various concentrations of an osmotic agent (usually dextrose). Ultrafiltration continues until the dialysis fluid becomes virtually isotonic, after which the rate that fluid is absorbed into the circulation exceeds that of the ultrafiltration induced by transcapillary hydrostatic pressure gradient alone. Net solute and water removal during peritoneal dialysis have been shown to be reduced by dialysis fluid absorption. Through these two processes, diffusion and osmotic ultrafiltration, appropriate quantities of solute metabolites and fluid need to be removed to maintain the patient's body fluid volumes and composition within appropriate limits.

There are various types of peritoneal dialysis therapies, including continuous ambulatory peritoneal dialysis (“CAPD”), automated peritoneal dialysis (“APD”), including tidal flow APD, and continuous flow peritoneal dialysis (“CFPD”).

CAPD is a manual dialysis treatment. The patient manually connects a drain tube to the patient's implanted catheter, allowing used dialysis fluid to drain from the peritoneal cavity. The patient then connects the indwelling catheter to a bag of fresh dialyzing fluid, infusing fresh dialyzing fluid through the catheter and into the patient. The patient disconnects the catheter from the fresh dialyzing fluid bag and allows the dialyzing fluid to dwell within the peritoneal cavity, wherein the transfer of waste, toxins and excess water takes place. After a dwell period, the patient repeats the manual dialysis procedure, for example, four times per day, each treatment lasting about an hour. Manual peritoneal dialysis requires a significant amount of time and effort from the patient, leaving ample room for improvement.

Automated peritoneal dialysis (“APD”) is similar to CAPD in that the dialysis treatment includes drain, fill, and dwell cycles. APD machines, however, perform the cycles automatically, typically while the patient sleeps. APD machines free patients from having to manually perform the treatment cycles and from having to transport supplies during the day. APD machines connect fluidly to the patient's implanted catheter, to a source or bag of fresh dialyzing fluid and to a fluid drain. APD machines pump fresh dialyzing fluid from the dialyzing fluid source, through the catheter, into the patient's peritoneal cavity and allow the dialyzing fluid to dwell within the cavity and the transfer of waste, toxins and excess water to take place. APD machines pump used dialysis fluid from the peritoneal cavity, through the catheter, to the drain. The APD machine cycler measures the amount of fluid infused and the amount removed to compute the net fluid removal. Treatment may begin with an initial patient drain to empty the peritoneal cavity of used dialysis fluid from a prior treatment. The cycler then performs a series of fill, dwell, and drain cycles, typically finishing with a fill cycle. A “last fill” occurs often at the end of APD, which remains in the peritoneal cavity of the patient until the next treatment.

Both CAPD and APD are batch type systems that send used dialysis fluid to a drain. Tidal flow systems are modified batch systems. With tidal flow, instead of removing all the fluid from the patient over a longer period of time, a portion of the fluid is removed and replaced after smaller increments of time.

Continuous flow peritoneal dialysis (“CFPD”) systems clean or regenerate used dialysis fluid instead of discarding it. The systems flow fluid into or out of the patient via a dialysis fluid loop. Dialyzing fluid flows into the peritoneal cavity through one catheter lumen and out another catheter lumen. The fluid exiting the patient passes through a reconstitution device that removes waste from the dialysis fluid, e.g., via a urea removal column that employs urease to enzymatically convert urea into ammonia. The ammonia is then removed from the dialysis fluid by adsorption prior to reintroduction of the dialyzing fluid into the peritoneal cavity. CFPD systems are more complicated typically than batch systems.

APD (including tidal flow) and CFPD systems can employ a pumping cassette. The pumping cassette typically includes a flexible membrane that is moved back and forth to push and pull dialysis fluid out of and into, respectively, the cassette.

Peritoneal dialysis requires the maintenance of an aseptic technique for connection because of the high risk of peritoneal infection. The risk of infection is particularly high due to the high number of exchanges of dialyzing fluid to which the patient is exposed.

Peritoneal dialysis generally requires large volumes of dialyzing fluid. Generally, at each application, or exchange, a given patient will infuse two to three liters of dialyzing fluid into the patient's peritoneal cavity. The fluid is allowed to dwell for, e.g., one to three hours, at which time it used dialysis fluid or effluent is drained out and exchanged for a new fresh fluid fill. Generally, multiple, e.g., four, exchanges are performed daily. Therefore, approximately 8 to 12 liters of dialyzing fluid may be required per day, seven days a week, 365 days a year for each patient.

Dialyzing fluids have traditionally been provided in sealed, heat sterilized form, ready for use. Peritoneal dialysis fluid is typically provided in one of three standard dextrose or glucose levels. The bags are being delivered to a patient's home as one liter to six liter bags with one or more of the different dextrose or glucose concentrations. Again, a normal daily consumption may be around 8 to 12 liters of dialysis fluid.

In light of above, several problems become apparent. Shipping and storage of the large volume of fluids and associated disposable sets is space consuming. Additionally, the use of multiple prefilled bags produces waste materials in the form of empty containers and packaging.

An improved peritoneal dialysis system and associated operating methodology is needed accordingly.

### SUMMARY

The present disclosure sets forth systems, sub-systems, methods and structures for preparing dialysis solution in a disposable bag or batch disposable. The dialysis solution may be created at the point of use, e.g., at the PD machine. PD fluid is delivered directly to the patient's peritoneal cavity. PD fluid therefore needs to have a level of sterilization suitable for being introduced into the patient's peritoneum. The PD dialysis fluid of the present disclosure accordingly replaces premixed and sterilized PD fluid prepared prior to delivery to the location of use, usually the patient's home.

Premade and delivered PD dialysis fluid is provided in sterilized bags sized from one to six liters, which are packed into boxes and delivered, e.g., monthly, for use to the patient's home. The boxes of fluid may be cumbersome and heavy for PD patients to handle, and consume a substantial area in a room of their homes. One drawback of premixed PD is the large amount of dialysis solution that is needed to perform the treatment. Normally, the supplies are delivered every other week, or once a month, and consume significant space in an apartment/house, often resulting in the patient being surrounded by medical supplies. The bags and boxes also produce a relatively large amount of waste disposed of on a weekly or monthly basis.

The abundance of medical supplies and dialysis solution at a patient's home may alter the “feel” or “atmosphere” of the patient's home and the patient may start to feel as if he or she is living in a hospital setting rather than their own home. The negative impact on the “feel” or “atmosphere” of the patient's home may negatively affect a patient's mood or mental health. Additionally, the abundance of medical supplies is a continuous reminder of the patient's health condition, which may further affect the patient's mood and make it more difficult for the patient to have a sense of normalcy. The presence of the medical supplies may also make friends or family uneasy when visiting the patient's home.

In addition to the amount of space the sterilized bags consume, premixed solution bags are heavy and may weigh between 2 to 6 kg each, which may make the bags difficult to move for elderly and sick patients. Transporting the premixed dialysis solution bags and making the numerous connections between the bags and the dialysis equipment is a constant physical burden for many PD patients.

Deliveries where fluid is provided in premixed sterilized bags, e.g., monthly, for use to the patient's home may result in the formation of Glucose Degradation Products (“GDPs”), which are toxic and can give rise to early or advanced glycation end products. GDPs are generated in PD fluids when glucose is heated (e.g., as under heat sterilization) under acidic conditions as well as subsequently during storage of the sterilized fluids.

The present systems and methods reduce significantly both the amount of dialysis solution stored and handled by PD patients and the amount of waste produced. Additionally, the present systems and methods may reduce or eliminate the formation of GDPs.

The overall system of the present disclosure in an embodiment may include three primary components, namely, a PD cycler, a water purifier and a disposable set operating with both the cycler and the water purifier. The PD cycler may for example include a scale and a peristaltic pump actuator that operates with the disposable set of the present disclosure. The disposable set in an embodiment includes a peristaltic pumping tube actuated by the peristaltic pump actuator and various tubes and connectors leading to and from the peristaltic pumping tube. As described in detail below, the disposable set in an embodiment also includes a heating/mixing container. The batch containers or disposable bags may be delivered and provided with a predetermined amount of a concentrate, such as a glucose or dextrose solution, which may be in the form of wet or dry concentrates.

The disposable set in various embodiments also includes at least one, and in one embodiment two, sterile, sterilizing grade filters placed in series with each other. The sterile, sterilizing grade filters may be pass-through filters with pores having average diameters suitable to produce sterile fluid, e.g., 0.22 micron, including the capability of removing endotoxins, resulting in water quality suitable for PD. The sterile, sterilizing grade filters provide the final stage of sterilization for the water that is used to mix with the one or more concentrate to provide a dialysis fluid suitable for PD.

The overall system may include a water purifier and multiple components leading to the water purifier. The water purifier outputs water and possibly water suitable for peritoneal dialysis (“WFPD”). The multiple components may include, for example, a water softener, a particulate pre-filter, a carbon filter, an ion-exchange resin cartridge and a regenerating salts cartridge. The components are provided with and/or located between the water purifier and a source of potable or drinkable water. A bacterial growth inhibiting agent container may also be fluidly connected to the water purifier. The water purifier may include water purification equipment, such as one or more reverse osmosis unit, an electrodeionization unit, and/or an ultraviolet (“UV”) light source and one or more pump to move water within the water purifier and one or more heater to heat the water within the water purifier. The water purifier may also include at least one reservoir for holding a quantity of water to be purified and for mixing with an anti-bacterial growth agent if provided. The water purifier may also include a deaerator for removing air from the water being purified. The water purifier may further include or operate with pretreatment equipment described above, e.g., a water softener module, connected to the patient's potable water supply.

The water purifier may in an alternative embodiment include one or more ultrafilter to help bring the water exiting the water purifier to a WFPD level. For example, multiple ultrafilters may be provided to bring the water exiting the water purifier to a WFPD quality level, wherein the sterile, sterilizing grade filters discussed above for the disposable set may not be needed or provided. In another embodiment, the water purifier includes a single ultrafilter, while the disposable set includes a single sterilizing filter, the combination of which brings the water to a level of sterilization suitable for being delivered to the patient's peritoneal cavity. In the embodiment in which the disposable set includes two or more sterile, sterilizing grade filters, no ultrafilter may be needed or provided in the water purifier. For redundancy, however, it is contemplated to provide one or more ultrafilter in the water purifier in combination with one or more sterile, sterilizing grade filter in the disposable set.

The PD cycler is in one embodiment configured to operate primarily electrically using electrically actuated solenoid fluid valves, e.g., energized open, de-energized closed for fail safe operation and an electrically operated peristaltic pump. The PD cycler may in an alternative embodiment by configured to operate a cassette of a disposable set pneumatically. Here, the PD cycler may include one or more positive pressure tank and one or more negative pressure tank. Electrically actuated pneumatic solenoid valves may be located between the pressure tanks and the disposable cassette. A control unit of the PD cycler may electrically control the solenoid valves to selectively allow positive or negative pneumatic pressure to reach the valves and pump chambers of the disposable cassette. Positive pressure is applied to close a valve of the cassette or to perform a pump-out or expel stroke at a pump chamber of the cassette. Negative pressure on the other hand is applied to open a valve of the cassette or to perform a pump-in or fill stroke at a pump chamber of the cassette.

As discussed herein, mixing is performed at least in part inside the heater/mixing container provided as part of the disposable set. The heater of the cycler is located beneath a heating pan into which the heater/mixing container is located. The heating pan and heater are provided on top of a weigh scale. The container is prepackaged with one or more concentrate that mixes with a precise amount of WFPD as measured by a weigh scale to form dialysis fluid. To mix the dialysis fluid, the peristaltic pump may circulate the WFPD and one or more concentrate from the chamber and back into the chamber multiple times. While this is occurring, the heater heats the dialysis fluid to body temperature, e.g., 37° C.

The PD cycler in any case operates in three phases, typically including a fill phase, a dwell phase, and a drain phase. The cycler may (i) pull fresh dialysis fluid from a disposable heater/mixing bag (may also be referred to as a batch container) and push the fresh dialysis fluid to the patient, and after the dwell phase the cycler may (ii) pull used dialysis fluid from the patient into a disposable container and may then push the used dialysis fluid to drain.

In an embodiment, the control unit of the cycler sends a wired or wireless signal to a water purifier requesting a desired amount of WFPD, upon receipt of which the water purifier prepares and supplies the requested amount of WFPD to the disposable bag or batch container. The cycler may mix fresh dialysis solution (described in detail below including a mixing sequence). The heater/mixing container may be placed directly on a heating pan and serve as the heater/mixing bag.

As mentioned above, the present overall system may prepare PD dialysis fluid at the point of use. To do so, the control unit may cause the cycler to pump precise amounts (e.g., as measured by a weigh scale) of WFPD into the heating/mixing container, which includes precise and pre-supplied amounts of at least one concentrate, such as a glucose and a buffer concentrate together, for mixing and forming a dialysis fluid having a sterilization level suitable for being delivered to the peritoneal cavity of the patient. Structures to aid the mixing are discussed below. The present system, even assuming that the resulting fluid has been mixed homogeneously, may still provide testing and monitoring. In one embodiment, the WFPD is tested using one or more sensor, e.g., a conductivity sensor, to ensure that the water is properly purified before being sent to the disposable heating/mixing container. Additionally, mixed dialysis fluid is also tested using one or more sensor, e.g., a conductivity sensor. The sensor readings for the WFPD as well as the mixed dialysis fluid or dialysis solution may be monitored by a control unit that also controls the cycler. For PD, the doctor typically prescribes a type of dialysis fluid to be used for treating a particular patient. Different PD dialysis fluids are typically differentiated by dextrose or glucose levels. For example, the assignee of the present disclosure provides different PD dialysis fluids having the following dextrose and glucose levels:
1.5% dextrose monohydrate (or glucose monohydrate)=1.36% anhydrous dextrose (or anhydrous glucose),
2.5% dextrose monohydrate (or glucose monohydrate)=2.27% anhydrous dextrose (or anhydrous glucose), and
4.25% dextrose monohydrate (or glucose monohydrate)=3.86% anhydrous dextrose (or anhydrous glucose).

The present system is also able to produce custom dextrose level finally mixed dialysis fluids optimized for the patient, e.g., somewhere between the approved levels of 1.5% and 4.25% dextrose. It is therefore contemplated to use one or more conductivity cell or sensor to confirm that the dialysis solution has been mixed to the correct proportions prescribed by the doctor or clinician. The conductivity cell may be part of the disposable set operable with the cycler. In another example, the conductivity cell may be part of the water purifier, wherein the information obtained from the conductivity cell may be monitored by or communicated to the cycler. The conductivity cell located in the water purifier may be reused, thereby removing cost in providing the conductivity cell with the cycler. In any case, when the cycler has completed its mixing, the cycler may send a sample of the mixture to the conductivity sensor, which reads the conductivity of the sample. The conductivity reading(s) may be analyzed to determine whether the dialysis solution is acceptable. For example, the system may report a “solution good” or “solution bad” result. If the dialysis solution is not acceptable, the system may produce an alert or alarm alarming or may discard the improperly proportioned dialysis fluid to drain.

When the conductivity cell is placed inside of the disposable set, the disposable set may be provided with conductivity probes that contact the dialysis fluid. The conductivity readings for any of the conductivity sensor embodiments discussed herein may be temperature compensated, and thus a temperature sensor, e.g., a thermistor or thermocouple, may be provided with any of the conductivity sensor embodiments described herein. Also, in any of the conductivity sensor embodiments discussed herein, the line leading to the conductivity sensor, e.g., the drain line or a sample line, may have a one-way valve, e.g., a duck-billed check valve, which helps prevent contaminants from migrating counter-flow up into the disposable cassette.

In light of the present disclosure, and without limiting the disclosure in any way, in a first aspect of the present disclosure, which may be combined with any other aspect, or portion thereof, a peritoneal dialysis system includes a water purifier; a disposable set including a water line in fluid communication with the water purifier, a drain line for draining from the disposable set, and a disposable container including at least one chamber, the disposable container including at least one concentrate in one of the at least one chamber, and the disposable container positioned and arranged to hold a dialysis fluid prepared by mixing water from the water purifier and the at least one concentrate; and a control unit in communication with at least one sensor, for detecting a first property of water from the water purifier and a second property of the dialysis fluid.

In a second aspect of the present disclosure, which may be combined with any other aspect, or portion thereof, the at least one sensor is a conductivity sensor.

In a third aspect of the present disclosure, which may be combined with any other aspect, or portion thereof, the first property and the second property are conductivities.

In a fourth aspect of the present disclosure, which may be combined with any other aspect, or portion thereof, the at least one sensor is housed within the water purifier.

In a fifth aspect of the present disclosure, which may be combined with any other aspect, or portion thereof, the at least one sensor is at least partially housed within the disposable set.

In a sixth aspect of the present disclosure, which may be combined with any other aspect, or portion thereof, the peritoneal dialysis system includes at least one of a weigh scale for weighing the disposable container or a heater for heating the dialysis fluid.

In a seventh aspect of the present disclosure, which may be combined with any other aspect, or portion thereof, the peritoneal dialysis system further includes a cycler configured to (i) mix water from the water purifier and the at least one concentrate to form a dialysis fluid and (ii) deliver the dialysis fluid to a patient.

In an eighth aspect of the present disclosure, which may be combined with any other aspect, or portion thereof, the cycler includes a heating pan configured to hold the disposable container and to heat an area within the disposable container.

In a ninth aspect of the present disclosure, which may be combined with any other aspect, or portion thereof, the control unit is part of the cycler.

In a tenth aspect of the present disclosure, which may be combined with any other aspect, or portion thereof, the control unit stores a look-up table with at least one setpoint value for at least one of the first property or the second property.

In an eleventh aspect of the present disclosure, which may be combined with any other aspect, or portion thereof, the control unit is programmed to compare a sensed value for at least one of the first property or the second property sensed by the at least one sensor to a setpoint value.

In a twelfth aspect of the present disclosure, which may be combined with any other aspect, or portion thereof, one of the at least one chamber of the disposable set is adapted for receiving used dialysis fluid from a patient drain.

In a thirteenth aspect of the present disclosure, which may be combined with any other aspect, or portion thereof, the disposable set includes a pumping tube for operation with a peristaltic pump.

In a fourteenth aspect of the present disclosure, which may be combined with any other aspect, or portion thereof, a peritoneal dialysis system includes a disposable set having a water line for fluid communication with a water source, a disposable container having an inlet and an outlet, the container including at least one concentrate, the disposable container positioned and arranged to hold a dialysis fluid prepared by mixing purified water from the water source and the at least one concentrate to form the dialysis fluid, a pumping line segment in fluid communication with the disposable container, a patient line downstream of the pumping line, and a mixing line segment in fluid communication with the disposable container and the pumping line; and a cycler including a pump positioned along the pumping line, the cycler configured to perform at least one of (i) a recirculation phase for mixing purified water with the at least one concentrate using the mixing line, (ii) a patient fill for delivering the dialysis fluid to a patient, (iii) a patient drain for removing used dialysis fluid from the patient, and (iv) moving drain fluid from the disposable container to a drain.

In a fifteenth aspect of the present disclosure, which may be combined with any other aspect, or portion thereof, the peritoneal dialysis system includes at least one sensor for confirming a property of at least one of the purified water or the dialysis fluid.

In a sixteenth aspect of the present disclosure, which may be combined with any other aspect, or portion thereof, the pump is a peristaltic pump operating with the pumping line segment.

In a seventeenth aspect of the present disclosure, which may be combined with any other aspect, or portion thereof, the cycler includes a weigh scale configured to weigh dialysis fluid delivered from the disposable container during the patient fill and the used dialysis fluid delivered to the disposable container during the patient drain.

In an eighteenth aspect of the present disclosure, which may be combined with any other aspect, or portion thereof, the cycler includes a heater configured to heat purified water and the at least one concentrate mixed during the recirculation phase.

In a nineteenth aspect of the present disclosure, which may be combined with any other aspect, or portion thereof, the cycler includes a pressure sensor positioned and arranged to measure a patient fill pressure of dialysis fluid delivered from the disposable container during the patient fill and a patient drain pressure of the used dialysis fluid delivered to the disposable container during the patient drain.

In a twentieth aspect of the present disclosure, which may be combined with any other aspect, or portion thereof, the cycler, when performing the recirculation phase of (i), is configured to cause the pump to pull a mixture of the purified water and the at least one concentrate from the outlet of the disposable container, through a downstream line segment, and into an upstream portion of the pumping line segment, and cause the pump to push the mixture of water and the at least one concentrate to a downstream portion of the pumping line segment through the mixing line segment, and into the disposable container through the inlet to form the dialysis fluid.

In a twenty-first aspect of the present disclosure, which may be combined with any other aspect, or portion thereof, the cycler, when performing the recirculation phase of (i), is configured to cause the pump to push the mixture of purified water and the at least one concentrate to a sensing line segment positioned between the pumping line segment and the mixing line segment.

In a twenty-second aspect of the present disclosure, which may be combined with any other aspect, or portion thereof, the cycler, when performing the patient fill of (ii), is configured to cause the pump to pull the dialysis fluid from the outlet of the disposable container, through a downstream line segment, and into an upstream portion of the pumping line segment, and cause the pump to push the dialysis fluid to a downstream portion of the pumping line segment and through the patient line.

In a twenty-third aspect of the present disclosure, which may be combined with any other aspect, or portion thereof, wherein the cycler, when performing the patient fill of (ii), is configured to cause the pump to push the dialysis fluid to a sensing line segment positioned between the pumping line segment and the patient line.

In a twenty-fourth aspect of the present disclosure, which may be combined with any other aspect, or portion thereof, wherein the cycler, when performing the patient drain of (iii), is configured to cause the pump to pull the used dialysis fluid from a patient, through the patient line, and through a downstream portion of the pumping line segment, and cause the pump to push the used dialysis fluid to an upstream portion of the pumping line segment, through a waste line segment to a drain compartment of the disposable container.

In a twenty-fifth aspect of the present disclosure, which may be combined with any other aspect, or portion thereof, wherein the cycler, when performing (iv) moving drain fluid from the disposable container to a drain, is configured to cause the pump to pull the used dialysis fluid from the drain compartment of the disposable container and push the used dialysis fluid to a house drain or terminal drain container.

In a twenty-sixth aspect of the present disclosure, which may be combined with any other aspect, or portion thereof, a disposable set includes a water line for fluid communication with a water source; a disposable container including a fresh dialysis fluid chamber including at least one concentrate, the fresh dialysis fluid chamber configured to hold fresh dialysis fluid prepared by mixing purified water delivered via the water line and the at least one concentrate to form the fresh dialysis fluid, and a drain chamber for receiving used dialysis fluid; at least one pumping line segment in fluid communication with the fresh dialysis fluid chamber and the drain chamber; and a patient line in fluid communication with the at least one pumping line segment.

In a twenty-seventh aspect of the present disclosure, which may be combined with any other aspect, or portion thereof, a connecting end of the water line is structured such that the connecting end is able to be unconnected and leak free during a treatment using the disposable container, the at least one pumping line segment, and the patient line.

In a twenty-eighth aspect of the present disclosure, which may be combined with any other aspect, or portion thereof, the disposable set includes a drain line in fluid communication with the drain chamber, and wherein a distal end of the drain line is structured such that the distal end is able to be unconnected and leak free during a treatment using the disposable container, the at least one pumping line segment, and the patient line.

In a twenty-ninth aspect of the present disclosure, which may be combined with any other aspect, or portion thereof, the disposable set includes a recirculation line in fluid communication with the fresh dialysis fluid chamber, the recirculation line positioned and arranged to aid in mixing purified water and the at least one concentrate in the fresh dialysis fluid chamber.

In a thirtieth aspect of the present disclosure, which may be combined with any other aspect, or portion thereof, the recirculation line is connected between the water line and the patient line.

In a thirty-first aspect of the present disclosure, which may be combined with any other aspect, or portion thereof, the recirculation line includes at least a portion of the at least one pumping line segment.

In a thirty-second aspect of the present disclosure, which may be combined with any other aspect, or portion thereof, a multi-chamber disposable container includes a first chamber including a fist concentrate; a second chamber including a second concentrate; a used dialysis fluid chamber for receiving used dialysis fluid; and plural fluid passages in fluid communication with the first chamber, the second chamber and the used dialysis fluid chamber.

In a thirty-third aspect of the present disclosure, which may be combined with any other aspect, or portion thereof, a method includes confirming a first property of water prepared by a water purifier; sending a first quantity of the water prepared by the water purifier to a disposable container that has multiple chambers, wherein the disposable container includes at least one concentrate in at least one of the multiple chambers; mixing the first quantity of water and the at least one concentrate in the disposable container to form a homogenous dialysis fluid; confirming a second property of the homogenous dialysis fluid; and performing at least one fill cycle using the homogenous dialysis fluid.

In a thirty-fourth aspect of the present disclosure, which may be combined with any other aspect, or portion thereof, at least one of the first or second properties is conductivity.

In a thirty-fifth aspect of the present disclosure, which may be combined with any other aspect, or portion thereof, mixing the first quantity of water and the at least one concentrate in the disposable container includes forming enough homogenous dialysis fluid for an entire treatment.

In a thirty-sixth aspect of the present disclosure, which may be combined with any other aspect, or portion thereof, the water purifier is located adjacent to a tap water source, and wherein the disposable container is located adjacent to the water purifier for at least one of (i) sending the first quantity of the water, (ii) mixing the first quantity of water and the at least one concentrate, and (iii) confirming the second property of the homogenous dialysis fluid.

In a thirty-seventh aspect of the present disclosure, which may be combined with any other aspect, or portion thereof, the disposable container is transported to a treatment location for performing the at least one fill cycle using the homogenous dialysis fluid.

In a thirty-eighth aspect of the present disclosure, which may be combined with any other aspect, or portion thereof, the method includes transporting the disposable container to a house drain after performing (i) the at least one fill cycle using the homogenous dialysis fluid and (ii) at least one drain cycle removing used dialysis fluid to the disposable container, and wherein at the house drain, removing the used dialysis fluid from the disposable container to the house drain.

In a thirty-ninth aspect of the present disclosure, which may be combined with any other aspect, or portion thereof, the house drain is located adjacent to the tap water source.

In a fortieth aspect of the present disclosure, which may be combined with any other aspect, or portion thereof, one of the chambers of the disposable container is adapted to receive used dialysis fluid, and wherein the method further includes pumping used dialysis fluid to the chamber for weighing after which the used dialysis fluid is removed from the chamber.

In a forty-first aspect of the present disclosure, which may be combined with any other aspect, or portion thereof, weighing the used dialysis fluid in the chamber is performed at a treatment location.

In a forty-second aspect of the present disclosure, which may be combined with any other aspect, or portion thereof, confirming the first property or the second property includes comparing sensor data to a first setpoint value for the first property and a second setpoint value for the second property.

In a forty-third aspect of the present disclosure, which may be combined with any other aspect, or portion thereof, any of the features, functionality and alternatives associated with any of FIGS. 1A to 11 may be combined with any of the features, functionality and alternatives associated with any of the other of FIGS. 1A to 11.

In light of the above aspects and the following description, it is accordingly an advantage of the present disclosure to provide an improved peritoneal dialysis system.

It is another advantage of the present disclosure to provide a peritoneal dialysis system that purifies water to a level suitable for use in preparing fresh dialysis fluid.

It is a further advantage of the present disclosure to provide a peritoneal dialysis system that prepares dialysis fluid having a sterilization level suitable for being delivered to the peritoneal cavity of the patient at the point of use safely.

It is still a further advantage of the present disclosure to provide a peritoneal dialysis system that mixes dialysis fluid online at the time of treatment to reduce disposable size and cost.

It is still another advantage of the present disclosure to provide a peritoneal dialysis system that effectively tests the proportional accuracy of dialysis fluid made at the point of use.

Moreover, it is an advantage of the present disclosure to provide a simplified dialysis fluid cycler that may use a peristaltic pump and electrically actuated pinch valves.

It is another advantage of the present disclosure to use conductivity feedback to ensure at least one of proper water purification and/or dialysis fluid mixing.

Further still, it is an advantage of the present disclosure to provide dialysis fluids having dextrose or glucose levels optimized for the patient.

Still further, it is an advantage of the present disclosure to provide dialysis fluid treatments that optimally provide different dextrose or glucose level dialysis fluids for different fill procedures of a same treatment.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1A is a front elevation view of one embodiment of a pre-filled dialysis solution bag.

FIG. 1B is a front elevation view of one embodiment of a disposable mixing/heating container according to an example embodiment of the present disclosure.

FIG. 2 is a front elevation view of one embodiment of a multi-chamber disposable mixing/heating container according to an example embodiment of the present disclosure.

FIG. 3 is a front elevation view of one embodiment of a disposable mixing/heating container according to an example embodiment of the present disclosure.

FIGS. 4A to 4C are schematic views of example embodiments of a dialysis system according to example embodiments of the present disclosure, including a balance chamber option.

FIG. 5A is a schematic view of an example proportioning phase according to an example embodiment of the present disclosure.

FIG. 5B is a schematic view of an example recirculation phase according to an example embodiment of the present disclosure.

FIGS. 6A to 6D illustrate fill, dwell, and drain cycles and a bag drain sequence according to example embodiments of the present disclosure.

FIG. 7A is a front elevation view of one embodiment of a disposable set according to an example embodiment of the present disclosure.

FIG. 7B is a front elevation view of one embodiment of a disposable set according to an example embodiment of the present disclosure.

FIG. 8 illustrates dimensions of the example disposable set according to an example embodiment of the present disclosure.

FIG. 9 is a process flow diagram illustrating one dialysis fluid mixing, dialysis fluid testing, and treatment method suitable for use with the system illustrated in FIGS. 4A and 4B.

FIG. 10 is a process flow diagram illustrating an example dialysis fluid mixing, testing, and treatment method suitable for use with any system described herein.

FIG. 11 is a front view of one embodiment for locating a conductivity cell with a line or tube of a disposable set.

### DETAILED DESCRIPTION

### Disposable Containers or Bags

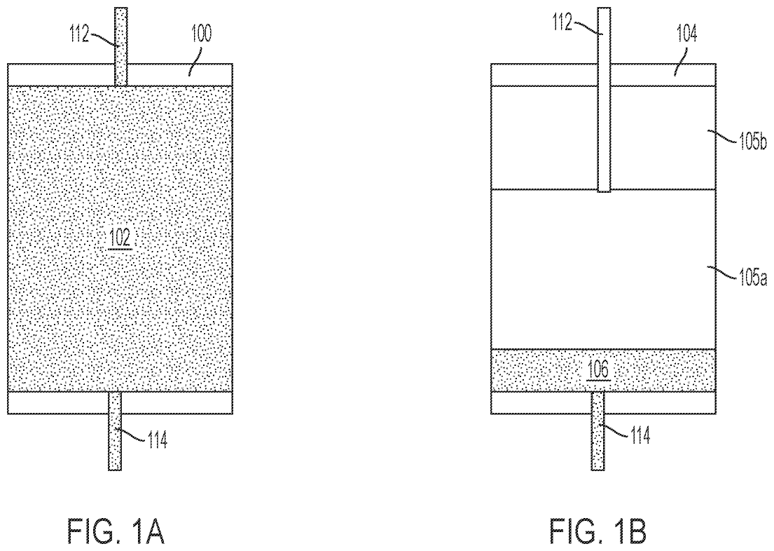

Referring now to the drawings and in particular to FIGS. 1A and 1, a typical dialysis solution container or bag 100 in FIG. 1A is pre-filled with dialysis solution 102 and delivered to a patient's home. Container or bag 100 may be a one liter bag or up to a six liter bag. Conversely, a heater/mixing container 104, as illustrated in FIG. 1B, may be delivered with only one or more peritoneal dialysis (“PD”) fluid concentrate(s) 106. Bag 100 and heater/mixing container 104, which may be generally referred to herein as bag 104, may include an inlet 112 and an outlet 114 or a single inlet/outlet. For example, a single disposable heater/mixing container or bag 104 with just concentrate(s) 106 (e.g., dry concentrate or liquid concentrate) may be delivered to the patient's home. This heater/mixing container 104 is much lighter than the premade and pre-filled dialysis solutions bags 100 that are typically shipped to patients. For example, the single heater/mixing container 104 may be almost empty (e.g., concentrate(s) 106 may amount to approximately 5% or less of the final dialysis solution 102) when shipped to the patient. Heater/mixing container or bag 104 can be sent to the patient either daily or weekly, with a weekly shipment weighing approximately 7 to 10 kg. The weekly shipment may, for example, include seven disposable heater/mixing containers 104 (e.g., disposable bags) that each account for a day's worth of treatment for APD. The seven disposable heater/mixing containers 104 may each result in approximately twelve liters of dialysis solution after mixing with water. The total prefilled/shipped concentrate volume may be around six liters. Heater/mixing container 104 may be divided into multiple chambers 105a, 105b creating a multiple-chamber container 104. For example, the heater/mixing container 104 may include a primary chamber 105a that holds concentrate(s) 106 and serves as a mixing and heating chamber and a second chamber that serves as a drain or effluent (used dialysis fluid) collection chamber (e.g., drain chamber 105b). In the multiple chamber configuration, it should be appreciated that the heating/mixing container 104 includes the appropriate inlets, outlets and other flow paths or connections that allow fluid communication with the primary chamber 105a, drain chamber 105b, inlet 112 and outlet 114 when performing filling, mixing or draining.

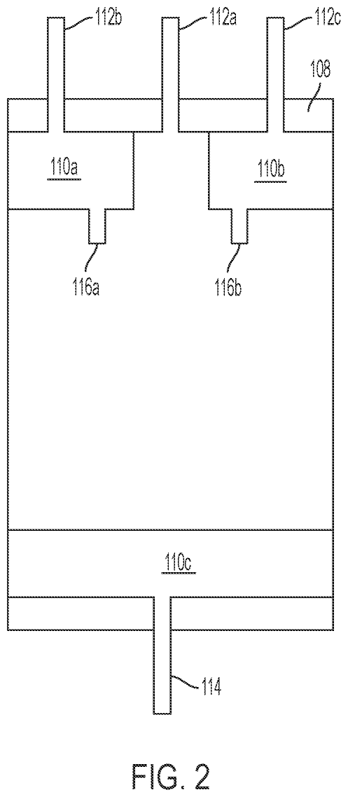

Disposable heating/mixing container(s) or bag(s) 104 may include different glucose concentrations and/or amounts for customizable or individualized therapies. For example, the shipment may include heating/mixing containers 104 with concentrates 106 configured (e.g., at 50% or greater dextrose) to create a final dialysis fluid after mixing, such as 1.5% dextrose final dialysis fluid, 2.5% dextrose final dialysis fluid, and 4.25% dextrose final dialysis fluid. In an alternative example, as illustrated in FIG. 2, the heating/mixing container 104 may be a variable heating/mixing container 108 that provides multiple alternative glucose concentrations. For example, the variable heating/mixing container 108 may include different chambers (e.g., chambers 110a, 110b and 110c, hereinafter referred to generally as chamber(s) 110) of concentrate(s) 106 that allow for a “four-in-one” alternative to produce a solution of 1.5%, 1.75%, 2% or 2.25% glucose. It should be appreciated that more or less chambers 110 may be implemented with different concentrates to produce different ranges of dialysis solutions 102. In one example illustrated in FIG. 2, chamber 110c may be associated with a base concentrate or a low concentration, e.g., 0.25% dextrose or glucose concentrate 106, assuming such concentrate may be mixed with that of other one or more chamber, chamber 110b may be associated with a 0.5% dextrose or glucose concentrate and chamber 110a may be associated with a 1.5% dextrose or glucose concentrate. In another (flipped) example, chamber 110a may be associated with a 0.25% dextrose or glucose concentrate 106, chamber 110b may be associated with a 0.5% dextrose or glucose concentrate, and chamber 110c may be associated with a 1.5% dextrose or glucose concentrate. It should be appreciated that the heating/mixing container 108 may include other concentrate arrangements (e.g., which concentrate 106 is associated with a particular chamber) and final dialysis fluid concentrations (e.g., 1.5%, 2.5%, 4.25% dextrose, etc.) than those illustrated above. The variable heating/mixing container 108 may include multiple inlets 112a-c and an outlet 114. For example, the final concentration of the dialysis solution 102 may be controlled by filling the variable heating/mixing container 108 through one or more of the inlets 112a to 112c. In the example illustrated in FIG. 2, chambers 110a and 110b have intermediate outlets 116a and 116b respectively. It is also contemplated that concentrate 106 may additionally include a desired amount of OCH.

By mixing the dialysis solution 102 (e.g., PD solution) on-site, a significant volume of dialysis solution 102 may be used per the physician's prescription without having to carry, move and connect multiple heavy pre-filled dialysis solution bags 100. As used herein, dialysis solution 102 may also be referred to as dialysis fluid 102.

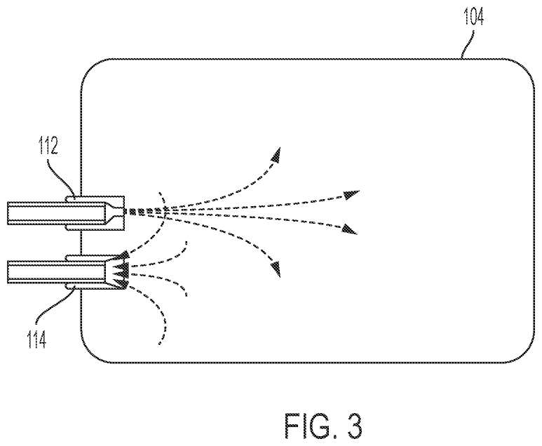

FIG. 3 illustrates another example of a disposable heating/mixing container or bag 104. Heater/mixing container 104 includes an inlet 112 and an outlet 114 that are adapted to improve mixing. For example, purified water for PD (“WFPD”) may be provided to the heater/mixing container 104 through an injector to the inlet 112, which may improve mixing. The outlet 114 may be positioned to further improve mixing, for example, by positioning the outlet 113 on an opposite end of disposable heating/mixing container or bag 104.

### Systems

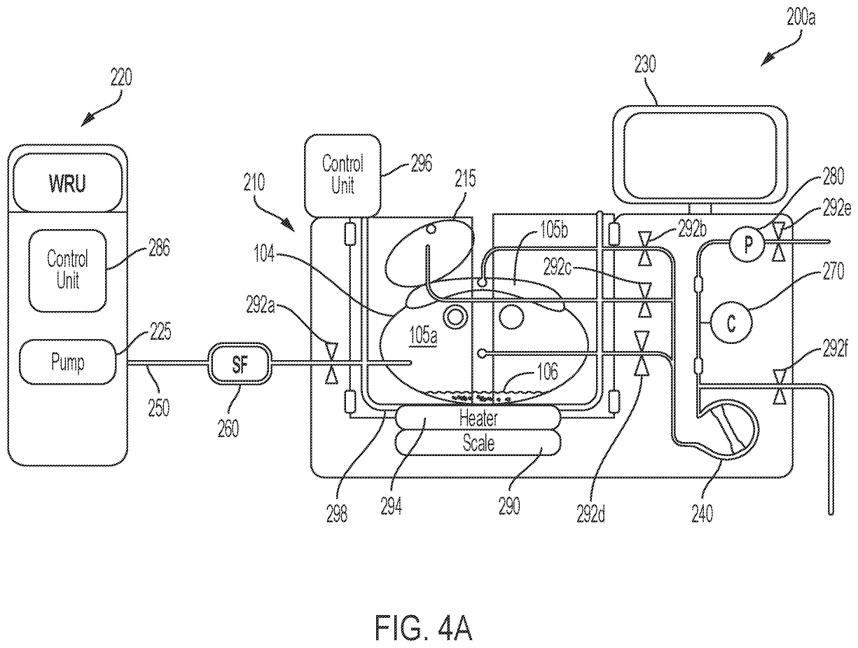

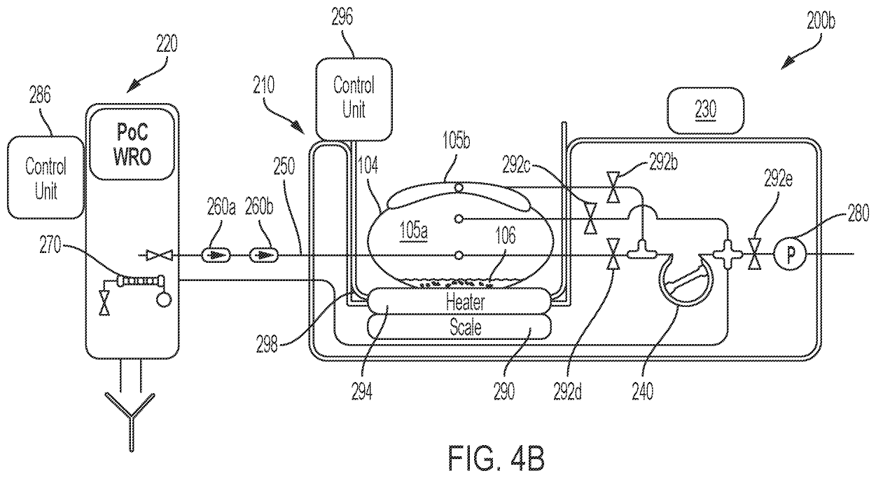

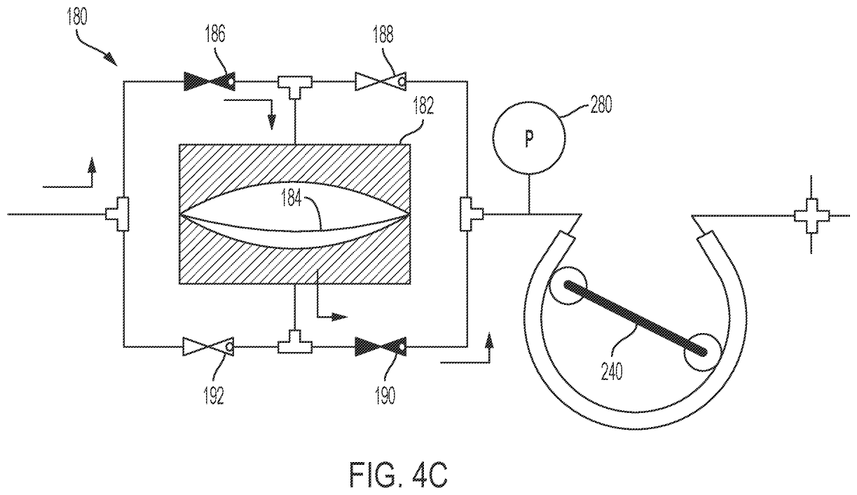

Referring now to FIG. 4A, one embodiment of a peritoneal dialysis system having point of use dialysis fluid production of the present disclosure is illustrated by system 200a. System 200a includes a cycler 210 and a water purifier 220. Water purifier may include a control unit 286 having at least one processor, at least one memory and a video controller operating with display device. Cycler 210 may include a control unit 296 having at least one processor, at least one memory and a video controller operating with display device 230, which may include a touch screen enabling display device 230 to also enter user information and provide a user interface. The control unit 296 may further include a wired or wireless transceiver for sending information to and receiving information from control unit 286 of water purifier 220. The control unit 286 of the water purifier 220 may further include a wired or wireless transceiver for sending information to and receiving information from cycler 210 or its associated control unit 296. Wired communication may be via Ethernet connection, for example. Wireless communication may be performed via any of Bluetooth™, WiFi™, Zigbee®, Z-Wave®, wireless Universal Serial Bus (“USB”), or infrared protocols, or via any other suitable wireless communication technology.

In an example, clean water may be produced on-site by water purifier 220 to dilute the concentrate(s) 106 in the heater/mixing container 104 to create the final dialysis solution 102 (e.g., PD solution). Clean water that meets the requirements of ultrapure water for dialysis (e.g., HD) should be sufficient for a PD therapy with one exception. The CFU requirement of the clean water may be set to zero for PD. Typically, for HD, the CFU limit is set to 0.1 CFU/ml for ultrapure water.

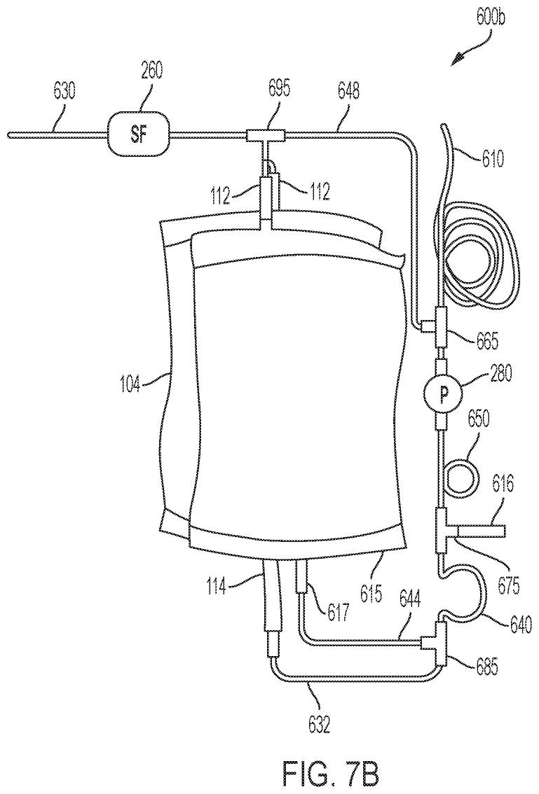

The cycler 210 may be programmed to prepare fresh dialysis solution 102 at the point of use including mixing the dialysis fluid, and pump the freshly prepared dialysis solution 102 to a patient, allow the dialysis solution 102 to dwell within the patient, then pump used dialysis fluid to a drain. Specifically, the cycler 210 of system 200a in FIG. 4A includes a peristaltic pump 240. The peristaltic pump 240 under control of control unit 296 may be bidirectional and circulate, e.g., back and forth, WFPD along with concentrate(s) 106, hereinafter referred to as an inhomogeneous mixture, within the lower chamber of the heater/mixing container 104 (e.g., the primary chamber 105a) for mixing and may also pump used fluid to a drain chamber (e.g., the drain chamber 105b illustrated in FIG. 1B) or a separate drain bag 215 (similar to drain bag 615 illustrated in FIG. 7B) when performing a drain cycle. After sufficient circulation of the inhomogeneous mixture, the inhomogeneous mixture becomes a homogeneous mixture of freshly prepared dialysis solution 102. Heater/mixing container 104 in the illustrated embodiment is placed by the patient or caregiver into a heating pan 298, which is located on top of heater 294. Although not illustrated, it is contemplated to provide one or more temperature sensor(s) located on or within the heating pan 298 so as to be able to estimate PD fluid temperature of the fluid residing within the heater/mixing container 104, and which outputs to control unit 296 for use as feedback to control heater 294.

System 200a may include a water line 250 that extends from water purifier 220 to cycler 210, such that the water from the water line 250 may be used to fill heater/mixing container 104 to mix with at least one concentrate(s) 106 located therein. WFPD may be supplied or pumped to the water line from pump 225 located within the water purifier 220. Alternatively, peristaltic pump 240 of cycler 210 may be configured to pull WFPD from water purifier 220 into heater/mixing container 104, such that water purifier 220 does not need a pump. The end of water line 250 of FIGS. 4A and 4B, and the ends of any of the other water lines discussed herein, which connects to water purifier 220 may be fitted with a one-way valve or check valve (not illustrated) that is oriented so as to allow purified water to flow in a direction from water purifier 220 to primary chamber 105a of heater/mixing container 104, but which prevents purified water from flowing in the opposite direction through water line 250 towards water purifier 220. The one-way valve or check valve accordingly automatically prevents purified water from leaking out of the connecting end of water line 250 when disconnected from water purifier 220, e.g., for the transport of cycler 210, including the accompanying disposable set, to a treatment location as discussed in connection with FIG. 10. A cap and/or clamp may be provided additionally or alternatively to the one-way valve or check valve for closing off the connecting end of water line 250 when disconnected from water purifier 220.

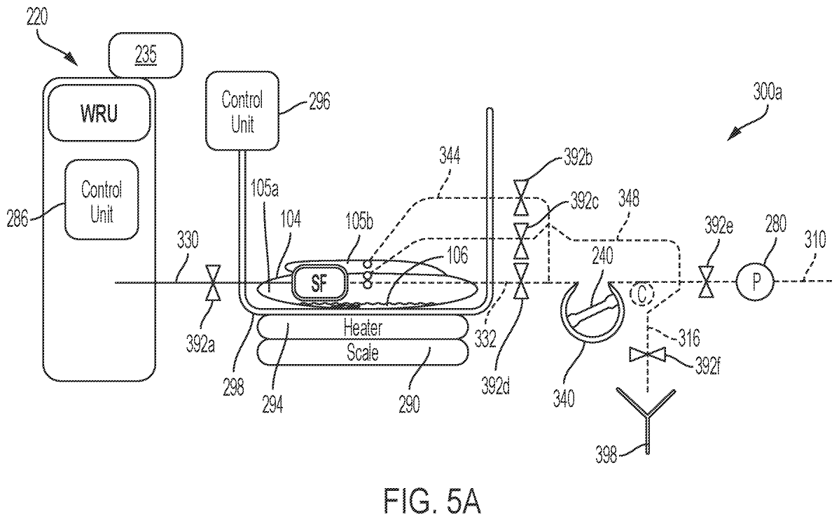

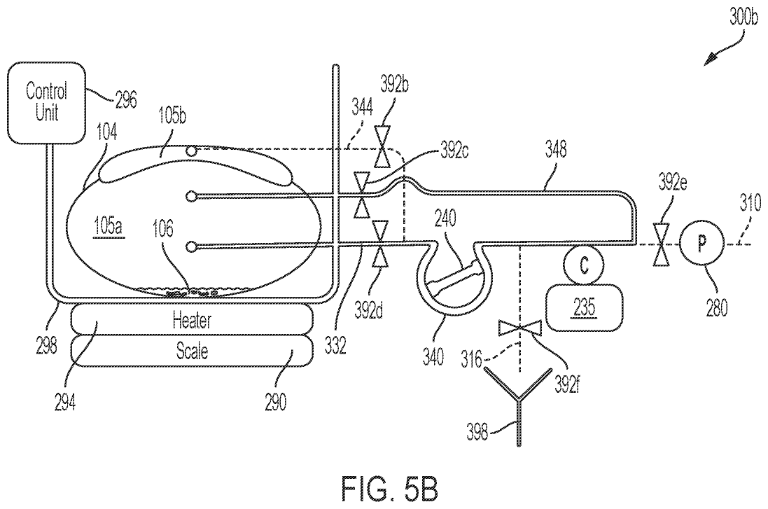

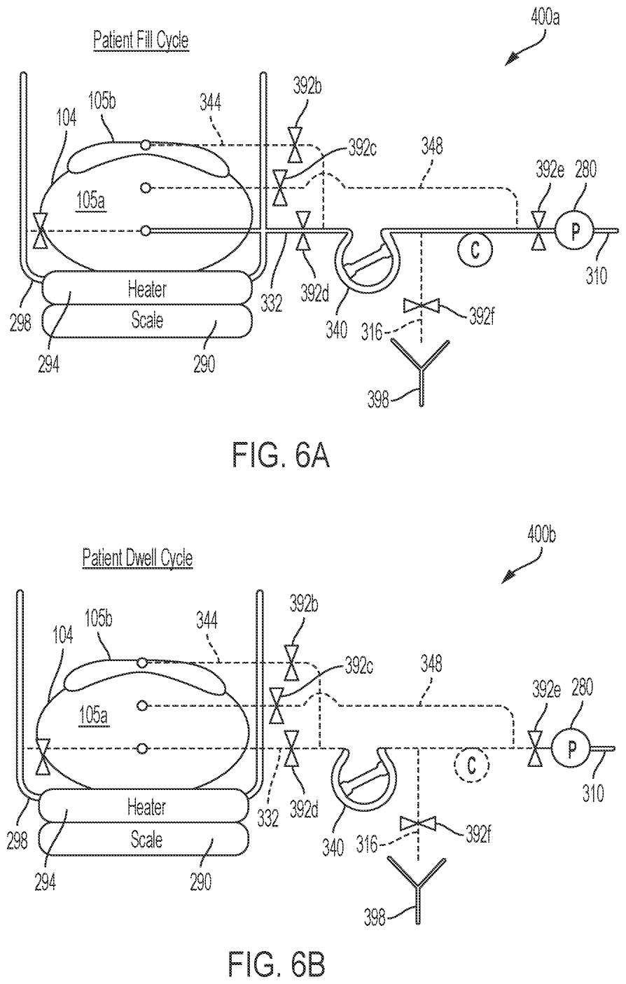

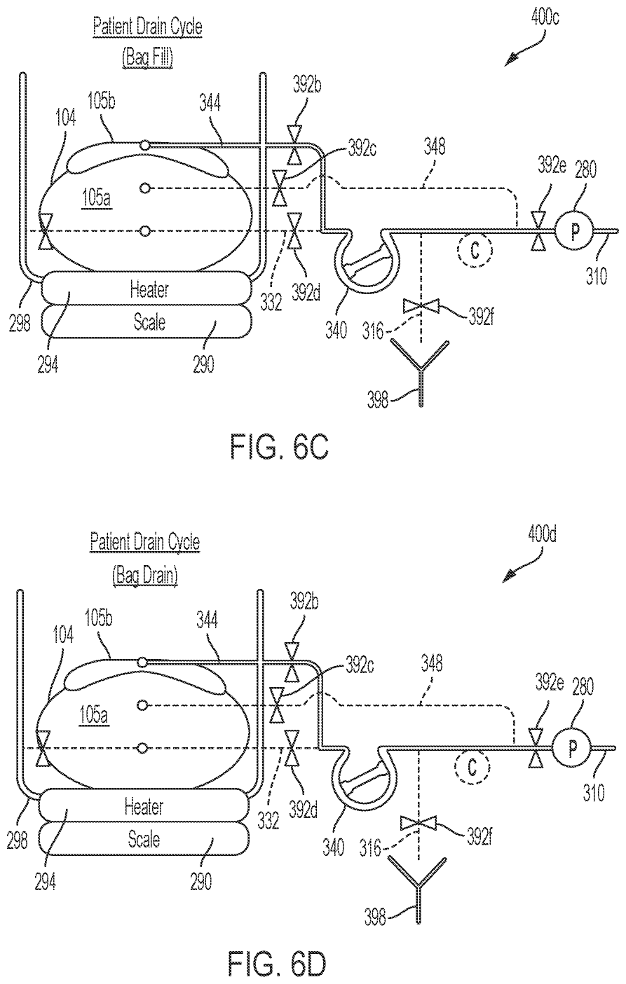

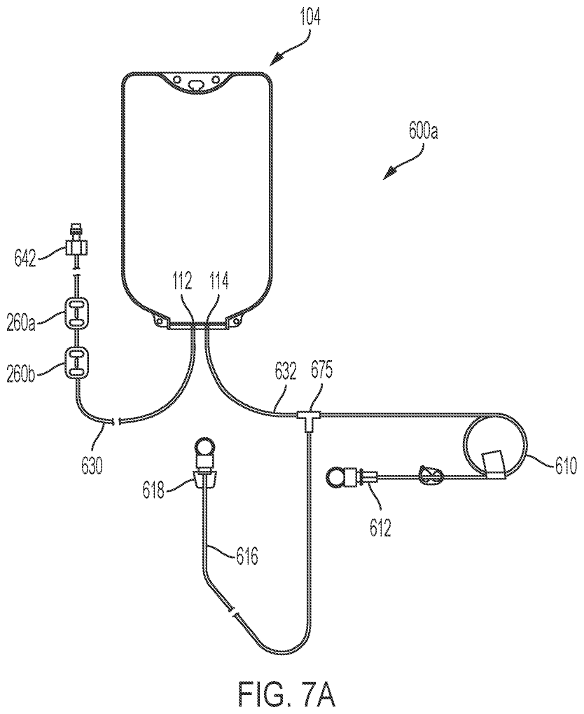

In order for the peristaltic pump 240 of the cycler 210 to pull WFPD from the water purifier 220, the arrangement of lines and flow paths may be altered to allow fluid communication between the peristaltic pump and the water purifier, for example another line connecting water line 250 with the peristaltic pump 240 and a return line leading back to the water purifier 220 may be implemented. In the illustrated embodiment, cycler 210 of system 200a includes a weigh scale 290 outputting weight signals to control unit 296, which closes inlet valve 292a when the correct amount of WFPD is added to the at least one concentrate(s) 106 located heater/mixing container 104. Cycler 210 of system 200a in the illustrated embodiment also include fluid valves 292b, 292c, 292d, 292e and 292f (described in more detail in relation to FIGS. 5A, 5B and 6A to 6D below). Valves 292a to 292f in the illustrated embodiment are electrically actuated pinch valves (e.g., solenoid pinch valves, rotational pinch vales, etc.) that either pinch a corresponding line or tube closed or open to allow fresh or used dialysis fluid to flow through the line or tube. In an embodiment, each of valves is under control of control unit 296 and is energized open and de-energized closed for fail safe operation.

Cycler 210 of system 200a in the illustrated embodiment also includes a heater 294 under control of control unit 296, which sits atop weigh scale 290 for heating the contents of heater/mixing container 104, e.g., to body temperature or 37° C. The water purifier 210 outputs water and possibly water suitable for peritoneal dialysis (“WFPD”). It should be appreciated that the water purifier 210 may be configured to deliver water at a temperature near or at 37° C. such that the need for heating is minimized, which may advantageously shorten the time to fully prepare the dialysis solution. To ensure WFPD, however, at least one sterile, sterilizing grade filter 260 is placed along water line 250. Sterile, sterilizing grade filter 260 may be a pass-through filter that does not have a reject line. Pore sizes for the sterilizing filter may, for example, be less than a micron, such as 0.1 or 0.2 micron. Suitable sterile, sterilizing grade filters 260 may, for example, be Pall IV-5 or GVS Speedflow filters, or be filters provided by the assignee of the present disclosure. In an embodiment, only one upstream or downstream sterilizing filter 260 is needed to produce WFPD, that is, water suitable for making dialysis solution 102 for delivery to the peritoneal cavity of a patient. Nevertheless, two sterile sterilizing grade filters 260 may be provided for redundancy in case one fails.

The disposable set operating with cycler 210 of system 200a in the illustrated embodiment includes a conductivity sensor or conductivity cell 270 outputting to control unit 296. For example, the conductivity cell 270 may be included in the disposable set patient line so that conductivity may be confirmed in the important line leading to the patient. The conductivity cell 270 may be formed between two fluid connectors on the disposable line set and may include an associated temperature sensor. The conductivity sensor 270 is used to test the mixed dialysis solution 102 to make sure the dialysis solution 102 has been mixed correctly to the prescribed formulation. The patient line of the disposable set of system 200a in the illustrated embodiment also includes a pressure measuring pod or pressure sensor 280, which outputs to control unit 296. The pressure sensor 280 is provided to make sure patient pressure is monitored and is used for controlling the speed and pressure of peristaltic pump 240 so that a pressure limit (e.g., +14 kPa or +2 psig for filling and −9 kPa or −1.3 psig for patient draining) is not met or exceeded.

Another example system 200b, which is illustrated via cycler 210 in FIG. 4B, includes much of the same structure (numbered the same) and functionality as system 200a of FIG. 4A, instead provides conductivity cell or conductivity sensor 270 in water purifier 220. The conductivity cell or conductivity sensor 270 may be used again to ensure the correct mixing of the dialysis solution 102 to a prescribed formulation. Prior to mixing, the WFPD may be flowed through the conductivity cell 270 and the water purifier 220 or its associated control unit 286 may take one or more conductivity reading from conductivity sensor 270 for the WFPD and either (i) compare the reading(s) with an expected reading for WFPD and send, wired or wirelessly, a “WFPD conductivity sensor reading good” or “WFPD conductivity sensor reading fails” output to control unit 296 of the cycler 210, which takes appropriate action, or (ii) sends the conductivity reading(s) wired or wirelessly to control unit 296 of the cycler 210, so that control unit 296 associated with the cycler 210 may determine, e.g., compare the reading to a look-up table, if the conductivity sensor reading is good or not and take appropriate action. The above conductivity confirmation procedure may be performed using any one or more fluid having a known conductivity. Furthermore, the conductivity sensor(s) or conductivity cell(s) may be calibrated using a fluid having a known conductivity before performing the conductivity confirmation procedure described above. Conductivity sensor 270 may therefore have a dual purpose of confirming proper WFPD and dialysis fluid mixing. However, in some instances other tuned conductivity sensor(s) or conductivity cell(s) may be used to confirm (1) proper WFPD and (2) proper dialysis fluid mixing. For example, the respective conductivity cell for confirming (1) proper WFPD may be tuned such that its measuring range is tuned for pure water (e.g., tuned to read conductivities less than one μS/cm) and the respective conductivity cell for confirming (2) proper dialysis fluid mixing may be tuned such that its measuring range is tuned for dialysis solution (e.g., tuned to read conductivities around 11.5 mS/cm).

Similar to system 200a, system 200b also includes a weigh scale 290 that ensures the correct amount of water is added to the heater/mixing container 104 for mixing with one or more concentrate(s) 106. System 200b as illustrated includes valves 292b to 292e, heater 294, control unit 296, heating pan 298 and other components numbered the same as system 200a.

Heating pan 298 holds the heater/mixing container 104, which may be a dual-chamber bag as illustrated in FIG. 1B, where a lower chamber has the concentrate(s) 106 for solution preparation. The heater/mixing container 104 may also include an upper chamber that serves as a drain chamber 105b. Alternatively, a separate container or bag may be used as a drain container or drain bag. The cycler 210 in both systems 200a and 200b includes peristaltic pump 240 that circulates WFPD along with concentrate(s) 106 in the lower chamber (e.g., inhomogeneous mixture) through the lower chamber of the heater/mixing container 104 for mixing, pumps fresh dialysis fluid to the patient, pumps used fluid from the patient to the drain chamber 105b, and pumps used dialysis fluid from the drain chamber 105b to a house drain or terminal drain container or bag. As noted above, lines and flow paths may be altered or additional liens and flow paths may be added to the systems 200a and 200b to allow fluid communication between the peristaltic pump and the water purifier, thereby allowing the peristaltic pump 240 to circulate water through the lower chamber of the heater/mixing container 104. Weigh scale 290 therefore weighs each patient fill and drain and therefore monitors the weight or volume, and corresponding mass or volume flowrate, of the whole treatment and enables control unit 296 to estimate a total amount of ultrafiltration (“UF”) removed from the patient at the end of treatment.

It should be appreciated that weigh scale 290 may be replaced by an alternative volume control mechanism, such as one or more balance chamber(s). For example, the cycler 210 may include a balance chamber type structure between peristaltic pump 240 and heater/mixing container 104 for volumetric monitoring and accuracy. The one or more balance chamber(s) in an embodiment include an internal membrane or sheet that flexes back and forth due to fluid pressure. FIG. 4C illustrates one possible balance chamber arrangement 180, which is placed directly adjacent peristaltic pump 240 in the illustrated embodiment. Balance chamber arrangement 180 may be placed on either side of peristaltic pump 240. In the illustrated implementation, balance chamber arrangement 180 operates under negative pressure pumping to the patient (left to right) and under positive pressure pumping from the patient (right to left). Balance chamber arrangement 180 includes a rigid chamber 182 within which internal membrane or sheet 184 flexes back and forth to meter a known and controlled volume of fresh or used dialysis fluid per each stroke. Valves 186 and 190 are opened together via control unit 296, while valves 188 and 192 are closed, to enable fresh dialysis fluid to flow in the direction of the arrows in FIG. 4C as membrane or sheet 184 moves downwardly. In a next half-stroke, valves 188 and 192 are opened together via control unit 296, while valves 186 and 190 are closed, to enable fresh dialysis fluid to flow through valves 192 and 188 as membrane or sheet 184 moves upwardly. A known volume of fresh or used dialysis fluid is metered through peristaltic pump 240 during each half-stroke. Control unit 296 counts each stroke and half-stroke and multiplies same by the known stroke volume to know how much fresh or used dialysis fluid has been delivered overall and to meet a prescribed overall delivery amount if provided (e.g., for a patient fill). Valves 186 to 192 may be checked for leaks prior to each patient fill and drain to ensure the accuracy of balance chamber arrangement 180. Pressure sensor 280 may be moved to the position shown in FIG. 4C or a second pressure sensor may be added to perform the valve leak checks and to monitor positive and negative pressure applied to membrane or sheet 184 during treatment.

In the illustrated embodiment, conductivity sensor 270 is located in a drain line of water purifier 210. As discussed above, conductivity cell or conductivity sensor 270 is used in system 200b to test the conductivity of the prepared dialysis solution 102. In one example, control unit 286 of the water purifier 210 records one or more conductivity reading from conductivity sensor 270 for the mixed dialysis solution 102 and either (i) compares the reading(s) with an expected reading for WFPD and sends, wired or wirelessly, a “mixed dialysis fluid reading good” or “mixed dialysis fluid reading failed” output to the control unit 296 of the cycler 210, which takes appropriate action, or (ii) sends the conductivity reading(s) wired or wirelessly to the control unit 296 of the cycler 210. Then, the control unit 296 of the cycler 210 may determine, e.g., compare the reading to a look-up table, if the mixed dialysis solution 102 reading(s) is good or not. The comparison may be to a range, e.g., within five percent of the setpoint conductivity.

In an example, best illustrated by FIG. 4B, a sample of mixed fluid (e.g., freshly mixed dialysis solution 102) may be sent to a drain 295 via the water device 220. As noted above, a conductivity sensor or conductivity cell 270 may be placed along the drain 395 (e.g., within a drain line) to test the prepared dialysis solution 102. Referring to FIG. 7B, the prepared dialysis solution 102 may travel from heater/mixing container 104, through the port 114 and pumping line segment 640, and out through the drain line 616 to the water purifier 220 via pumping action from pump 240. Once at the water purifier 220, the dialysis solution 102 may be sent to the drain 295 and corresponding conductivity cell (e.g., conductivity cell 270) for testing.

The distal ends of the drain lines of FIGS. 4A and 4B, and the ends of any of the other drain lines discussed herein, may be fitted with a one-way valve or check valve (not illustrated) that is oriented so as to allow used dialysis fluid to flow in a direction towards the drain only when the distal end of the drain line is pressurized enough to overcome the closing force (e.g., applied via a spring) of the one-way valve or check valve. For example in FIG. 4A, when drain valve 292f is closed, the distal end of the drain line does not see any pressure applied via peristaltic pump 240, so that the one-way valve or check valve remains closed, preventing leakage from the end of the drain line. When drain valve 292f is opened, the distal end of the drain line sees pressure applied via peristaltic pump 240, so that the closing force of the one-way valve or check valve is overcome, opening the valve and allowing used dialysis fluid to be pumped to drain. The one-way valve or check valve accordingly automatically prevents used dialysis fluid from leaking out of the distal end of the drain line during treatment. A cap and/or clamp may be provided additionally or alternatively to the one-way valve or check valve for closing off the distal end of the drain line.

System 200b as with system 200a includes a water fill line 250 having at least one sterilizing filter. In the illustrated example, water fill line 250 may include two sterile sterilizing grade filters 260a, 260b, hereinafter referred to generally as filter(s) 260. Similar to system 200a, the filter(s) 260 of system 200b may be a pass-through filter that does not have a reject line. Pore sizes for the sterilizing filter may, for example, be less than a micron, such as 0.1 or 0.2 micron. Filters 260 may have a throughput of approximately 200 ml/min. However, depending on the choice of filter(s), the throughput may be different. In the case with smaller filter(s) having a low throughput (e.g., 200 ml/min), when the goal is to produce, e.g., fifteen liters of dialysis solution 102 for an entire treatment, producing such an amount of dialysis solution 102 takes a considerable amount of time, which requires a considerable amount of preparation. In such case, the preparation may be started at a time in advance of the start of treatments to ensure that the batch is ready to use at the beginning of treatment. Preparation of the dialysis solution 102 may be started prior to treatment for a first patient fill and continue during patient dwells for subsequent patient fills. In an example, preparation of the dialysis solution 102 during patient dwells may involve multiple containers 104.

It should be appreciated that systems 200a and 200b may be relatively simple from a hardware standpoint and use a single electrically actuated peristaltic pump and electrically actuated solenoid valves. The relatively simple hardware leads to a relatively simple disposable set. It should be appreciated however that systems 200a and 200b, and any other system described herein are not limited to electromechanical actuation and may be actuated in an alternative manner, e.g., via pneumatic actuation. The following equipment may be provided for such a pneumatic pumping system, including but not limited to (i) one or more positive pressure reservoir, (ii) one or more negative pressure reservoir, (iii) a compressor and a vacuum pump each under control of the control unit 296 associated with the cycler 210, or a single pump creating both positive and negative pressure under control of the control unit 296 associated with the cycler 210, for providing positive and negative pressure to be stored at the one or more positive and negative pressure reservoirs, (iv) plural pneumatic valve chambers for delivering positive and negative pressure to plural fluid valve chambers, (v) plural pneumatic pump chambers for delivering positive and negative pressure to plural fluid pump chambers, (vi) plural electrically actuated on/off solenoid pneumatic valves under control of the control unit 296 associated with the cycler 210 located between the plural pneumatic valve chambers and the plural fluid valve chambers, (vii) plural electrically actuated variable orifice pneumatic valves under control of the control unit 296 associated with the cycler 210 located between the plural pneumatic pump chambers and the plural fluid pump chambers, (viii) a heater under control of the control unit 296 associated with the cycler 210 for heating the dialysis solution 102 as it is being mixed in one embodiment, and (viii) an occluder under control of the control unit 296 associated with the cycler 210 for closing the patient and drain lines in alarm and other situations.

### Dialysis Fluid Mixing Control

Systems 200a and 200b also include a display device 230. The display device 230 is configured to display to the patient or caregiver that the quality of water produced on-site is proper and that the correct mixing of the WFPD and concentrate(s) 106 to form the final dialysis solution 102 has occurred. One or more control unit 286, 296 may be configured to monitor, obtain and analyze information obtained from the conductivity sensor(s) or conductivity cell(s) 270 described above with respect to systems 200a and 200b. In another example, fluid mixing control may be provided by a separate monitoring device, which may include the conductivity sensor or conductivity cell(s) 270 described above. The separate monitoring device may be modular or removable such that the monitoring device may be used with various components of systems 200a and 200b. As described in more detail below, during the proportioning phase, the separate monitoring device may be attached to an on-site water cleaning unit, such as water purifier 220 to obtain, monitor and analyze conductivity data about the WFPD and/or prepared dialysis solution 102. The monitoring device may also analyze conductivity data or other data about any mixture existing between the WFPD and the fully prepared dialysis solution 102. Based on the analysis of intermediate mixtures or solutions, the monitoring device may trigger adding more water to correct one or more concentrations of a prepared dialysis solution 102. Additionally, during the recirculation phase, the separate monitoring device may be attached to the cycler or disposable set to obtain, monitor and analyze conductivity data of the prepared dialysis solution 102.

In an example, control unit 286 and/or 296 and/or the separate monitoring device stores a look-up table with setpoint values for a sensed property (e.g., conductivity). The control unit or separate monitoring device may be programmed to compare a sensed value for the property sensed by a sensor, such as a conductivity sensor or conductivity cell 270, to a currently prescribed one of the setpoint values stored in the look-up table. Additionally, the setpoint value may be used for comparison to ensure that the measured conductivity corresponds to a desired formulation for the dialysis solution 102.

### Conductivity Cell

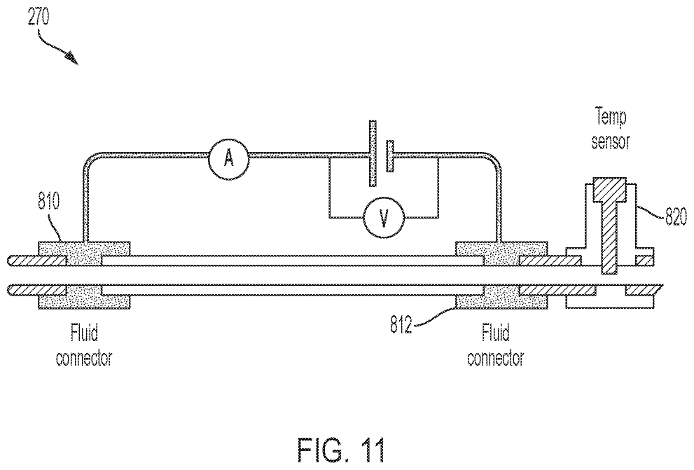

Referring now to FIG. 11, an example conductivity cell 270 is illustrated. The conductivity cell 270 may be positioned along one of the line segments, e.g., patient line, of the disposable set operating with a cycler of the present disclosure. Conductivity cell 270 may include two conducting fluid connectors 810, 812 (or fluid connectors with associated conducting electrodes) with a fluid line segment positioned therebetween. The conducting fluid connectors 810, 812 may be configured to measure conductivity by allowing a voltage (V) to be applied across the conducting connectors or conducting electrodes and allowing the corresponding current (A) to be measured at control unit 296, or vice versa. Additionally, the conductivity cell 270 may include a temperature sensor contact 820 leading to a temperature sensor, the output of which is used by control unit to compensate the conductivity reading accordingly. In one embodiment, conducting fluid connectors 810, 812 are disposable along with the disposable set and are spaced apart a distance to match that of two reusable contacts (not illustrated) provided at the cycler. The reusable contacts are placed in electrical communication with voltage source or reader (V) and current source or reader (A) located within the cycler. Temperature sensor contact 820 may also be disposable and be positioned to mate with reusable thermocouple leads in one example.

### Disposable Set

Referring now to FIG. 7A, an example embodiment of disposable set 600a is illustrated. Disposable set 600a may be mated to cycler 210 to move fluid within the disposable set 600a, e.g., to mix dialysis solution 102 as discussed herein. Disposable set 600a in the illustrated embodiment includes a patient line 610 that extends from connector 675 and terminates at a patient line connector 612. Conductivity cell 270 discussed above in connection with FIG. 11 may, for example, be located along patient line 610. Disposable set 600a may also include a drain line 616 that extends from connector 675 and terminates at a drain line connector 618. In an example, the drain line connector 618 may connect removeably to a drain connector associated with the water purifier 220, e.g., to allow freshly mixed dialysis fluid to be tested at the water purifier. Disposable set 600a may also include an upstream water line segment 630 that extends to an inlet 112 of a heater/mixing container 104. Downstream line segment 632 extends from an outlet 114 of the heater/mixing container 104 to connector 675. A water line connector 642 may be removeably connected to a water outlet connector of the water purifier 220. Upstream water line segment 630 may be water line 250 of FIGS. 4A and 4B. In another example, upstream water line segment 630 may be connected to water line 250 of FIGS. 4A and 4B. As mentioned previously, sterile sterilizing grade filter(s) 260a and 260b may be placed in upstream water line segment 630.

The line segments and components of disposable set 600a may correspond to line segments and components illustrated in FIGS. 5A, 5B, and 6A to 6D. For example, patient line 610 may correspond to patient line 310, drain line 616 may correspond to drain line 316, upstream water line segment 630 may correspond to upstream water line segment 330, etc. During use, water may be provided to inlet 112 of heating/mixing container 104 through upstream water line segment 630. Prepared dialysis solution 102 in heating/mixing container 104 may be passed through outlet 114 of heating/mixing container 104, through downstream line segment 632, and through patient line 610 to the patient. Used dialysis fluid may be sent back to heating/mixing container 104 through patient line 610, downstream line segment 632 and back through the outlet 114 of heating/mixing container 104. Then, the used dialysis fluid may be sent to the drain by passing the fluid through outlet 114, through downstream line segment 632 and through drain line 616 to the drain. Conversely, used dialysis fluid may be sent directly from the patient to the drain without filling the heating/mixing container 104.

Referring now to FIG. 7B, an example embodiment of an alternative disposable set 600b is illustrated. Disposable sets 600a and 600b may be generally referred to herein as disposable set 600. Disposable set 600b may be mated to cycler 210 to move fluid within the disposable set 600b, e.g., to mix dialysis solution 102 as discussed herein. Disposable set 600b in the illustrated embodiment includes a patient line 610 that extends from connector 655 and terminates at a patient line connector (not pictured). Disposable set 600b may also include a sensing segment 650 extending between connector 665 and connector 675, which may include a pressure sensor 280, a conductivity sensor or conductivity cell 270, or both the pressure sensor 280 and the conductivity cell 270. Specifically, conductivity cell 270 discussed above in FIG. 11 and/or the pressure sensor 280 may, for example, be located along patient line 610 (e.g., within the sensing segment 650). Additionally, disposable set 600b may also include a drain line 616 that extends from connector 675 and terminates at a drain line connector (not pictured). Similar to disposable set 600a, the drain line connector may connect removeably to a drain connector associated with the water purifier 220. Disposable set 600b may also include an upstream water line segment 630 that extends to a connector 695 or to an inlet 112 of a heater/mixing container 104. Downstream line segment 632 extends from an outlet 114 of the heater/mixing container 104 to connector 685. Additionally, disposable set 600b may include a mixing line segment 648 positioned between connectors 665 and 695 and a pumping line segment 640 positioned between connectors 685 and 675. In the illustrated embodiment, disposable set 600b also includes a drain bag 615 with an inlet/outlet 617 connected to a waste line segment 644, which terminates at connector 685.

Similar to disposable set 600a, the line segments and components of disposable set 600b may correspond to line segments and components illustrated in FIGS. 5A, 5B, and 6A to 6D. During use, water may be provided to inlet 112 of heating/mixing container 104 through upstream water line segment 630. To assist with mixing and preparing dialysis solution (e.g., during recirculation phase 300b), partially mixed dialysis solution may be pumped through outlet 114 of heating/mixing container 104, through downstream line segment 632, through pumping line segment 640 (optionally through sensing line segment 650 if present), through mixing line segment 648, and back to heating/mixing container 104 through inlet 112. To deliver prepared dialysis solution 102 to a patient, the dialysis solution 102 in heating/mixing container 104 may be passed through outlet 114 of heating/mixing container 104, through downstream line segment 632, through pumping line segment 640 (optionally through sensing line segment 650 if present), and through patient line 610 to the patient.

Used dialysis fluid may be sent from the patient to drain bag 615. For example, used dialysis fluid may be passed through patient line 610 (optionally through sensing line segment 650 if present), through pumping line segment 640, through waste line segment 644, and through the inlet/outlet 617 to the drain bag 615. To dispose of the used dialysis fluid, the spent fluid from drain bag 615 may be passed back through inlet/outlet 617, through waste line segment 644, through pumping line segment 640 and through drain line segment 616 to the drain. Any of the disposable sets discussed herein, including disposable sets 600a and 600b, may be made of one or more plastic, e.g., polyvinylchloride (“PVC”) or a non-PVC material, such as polyethylene (“PE”), polyurethane (“PU”) or polycarbonate (“PC”).

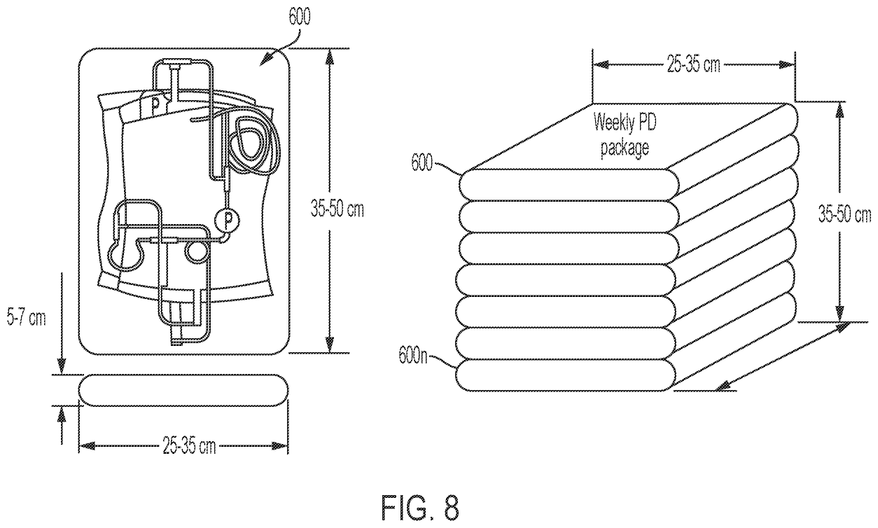

FIG. 8 illustrates example dimensions of one embodiment of a packaged and shipped disposable set 600 along with the dimensions of an example pack of a weekly shipment of the disposable sets 600 (e.g., up to 600n disposable sets). Disposable sets 600 may be sterilized individually, e.g., via gamma or ethylene oxide, or in the pack via steam sterilization.

### Filling and Recirculation

During the fill process or proportioning phase 300a as illustrated in FIG. 5A, the heater/mixing container 104 is almost empty (e.g., just contains concentrate(s) 106, which is approximately 5% of the final volume), then the heater/mixing container 104 is filled with a metered amount of WFPD. As illustrated in FIG. 5A, water from a water purifier 220 may pass through an upstream water line segment 330 to an inlet 112 of the heater/mixing container 104. The objective of the proportioning phase is to dilute the concentrate(s) 106 in the heater/mixing container 104 to create the final dialysis solution 102. In the illustrated embodiment, during the proportioning phase 300a, a separate monitor 235 is attached to an on-site water cleaning unit, such as water purifier 220. For example, the separate monitor 235 may obtain, monitor and analyze conductivity data from the conductivity cell 270 built into the water purifier 220. With a separate monitor 235, the water purifier 220 may include a connection port that the monitor 235 is adapted to connect to. In another example, the separate monitor 235 may have its own associated conductivity cell 270. Specifically, the separate monitor 235 may be used to ensure that the water provided to the heater/mixing container 104 has the expected conductivity associated with WFPD. For example, the separate monitor 235 may be modular or removable such that the display device 230 can be used with various components of systems 200a and 200b.

Scale 290 may be used with control unit 296 to control inlet valve 392a (or similarly inlet valve 292a of FIG. 4A) to the heater/mixing container 104 to ensure the correct amount of WFPD is added for mixing. During the proportioning phase 300a, control inlet valve 392a is open while fluid valves 392b to 392f are closed. WFPD is added to the container 104, through the upstream water line segment 330, from a pump or pumping unit of the water purifier 220. Since the pump or pumping unit of the water purifier 220 is not connected to the patient, the disposable container or heating/mixing container 104 may be filled quickly and at a higher pumping pressure. Once the prescribed amount of WFPD has been added to heater/mixing container 104, valve 392a between the disposable heating/mixing container or bag and water purifier 220 is closed.

A recirculation phase 300b, as illustrated in FIG. 5B, takes place next, which mixes the concentrate(s) 106 and WFPD within heater/mixing container 104 to make the final dialysis solution 102 homogenous. For example, after the heating/mixing container 104 is filled with WFPD, inlet control valve 392a is closed and fluid valves 392c and 392d are opened. The contents of the disposable container or heating/mixing container 104 may be passed from the outlet 114 of the heating/mixing container 104 through a downstream line segment 332 and open control valve 392d, through a pumping line segment 340 associated with via peristaltic pump 240, through a mixing line segment 348 and open control valve 392c, and back to the disposable container or heating/mixing container 104. During the recirculation phase, fluid valves 392a, 392b, 392e and 392f are closed. Peristaltic pump 240 may be operated in both clockwise and counterclockwise directions during recirculation to promote mixing.

Recirculation phase 300b may be done either as a separate phase or done simultaneously with the proportioning phase 300a. By performing the recirculation phase 300b simultaneously with the proportioning phase 300a, the mixing of the water and concentrate(s) 106 may be close to complete when the water filling phase is finished. Typically, the recirculation phase 300b continues for some time after the proportioning phase 300a to ensure that the dialysis solution 102 is properly mixed. The recirculation phase 300b may use a peristaltic pump 240 that moves on high speed to improve mixing and making the dialysis solution 102 homogenous. Additionally, moving the peristaltic pump 240 at higher speeds advantageously reduces mixing times. Referring back to FIG. 3, water (e.g., WFPD) may be added to the heater/mixing container 104 through an injector or the contents may be recirculated via the injector to further improve mixing. Alternatively, instead of WFPD, an inhomogeneous mixture of the WFPD along with concentrate(s) 106 or the dialysis solution 102 (dependent on preparation phase) may be added to the heater/mixing container 104. The peristaltic pump 240 or other pumping mechanisms associated with the cycler 210 may pull the dialysis solution 102 from the heater/mixing container 104 and push dialysis solution 102 back to the heater/mixing container 104 to ensure proper mixing. The dialysis solution 102 may also be passed through a separate heater/mixing bag (not pictured) to provide additional mixing. In an example, the separate heater/mixing bag may also be positioned on heater 294 to heat the contents therein.

During the recirculation phase 300b, the display device 230 displays pertinent parameters (or separate monitor 235 may be attached to the cycler 210). As noted above, the separate monitor 235 may obtain, monitor and analyze conductivity data from the conductivity cell 270 built into the cycler 210 (e.g., as part of cycler 210, peristaltic pump 240, disposable set, etc.). In another example, the separate monitor 235 may have its own associated conductivity cell 270. Even though one example of the conductivity cell 270 is illustrated in FIG. 11, the conductivity cell 270 may a reusable conductivity cell 270 that is the same as or similar to the conductivity cell(s) 270 used in hemodialysis machines. The separate monitor 235 may adapted to be removeably attached to both the water purifier 220 and the cycler 210, such that the separate monitor 235 may be used to ensure that the water provided to the heater/mixing container 104 has the proper conductivity and that the final dialysis solution 102 has the proper conductivity. In one embodiment, the proportioning phase 300a and recirculation phase 300b are intended to be performed only once per treatment session to mix WFPD and concentrate(s) 106 into to form enough dialysis solution 102 for an entire treatment. At the end of the recirculation phase 300b, the heater/mixing container 104 may contain up to fifteen liters of dialysis solution 102 ready to use over multiple patient fills. The proportioning phase 300a and the recirculation phase 300b are intended to be performed once per treatment session to mix the dialysis fluid to a homogenous solution suitable as dialysis solution 102 (e.g., PD fluid). At the end of the recirculation phase, the heater/mixing container 104 will have 15 liters of dialysis solution 102, ready to use.

It should be appreciated that the filling and preparation sequence may be performed in several steps. For example, filling the heater/mixing container 104 with approximately 95% of WFPD, then recirculating and measuring conductivity. Based on the measured conductivity, additional WFPD may be added to the heater/mixing container 104 (e.g., the heater/mixing container 104 is top-filled) and another round of recirculating and confirming conductivity of the fully prepared dialysis solution 102 with another conductivity measurement. Once conductivity is confirmed, the water purifier 220 may be shut off completely.

### Patient Fill, Patient Dwell, Patient Drain, Bag Drain

Once the recirculation phase 300b is complete and the conductivity of the dialysis solution 102 is confirmed with the display device 230, the final dialysis solution 102 may be used for treatment via an APD or CAPD patient fill cycle 400a, patient dwell cycle 400b, a patient drain cycle (bag fill) 400c and a bag drain procedure 400d as illustrated in FIGS. 6A, 6B, 6C and 6D, respectively. In an example, each dwell cycle 400b may be “individualized” to provide novel treatments for improved UF utilization in PD patients. Treatment may alternatively begin with an initial drain of the patient, which is weighed by weigh scale 290.

During a patient fill cycle 400a of FIG. 6A, the dialysis solution 102 is pumped from the disposable heating/mixing container or bag 104 to the patient's peritoneal cavity. Control unit 296 causes inlet control valve 392a is closed. Additionally, fluid valves 392b, 392c and 392f are closed, while fluid valves 392d and 392e are opened. For example, dialysis solution 102 from disposable heating/mixing container or bag 104 may be passed from the outlet 114 of the heating/mixing container 104 through the downstream line segment 332 and open control valve 392d, through a pumping line segment 340 associated with the peristaltic pump 240, and through a patient line 310 and open control valve 392e to the patient. Fill pressure is monitored by pressure sensor 280, which reads out to control unit 296, which in turn operates peristaltic pump 240 so as to create a safe patient fill pressure. Weigh scale 290 monitors the amount of fresh fluid delivered to the patient, so that control unit 296 can stop the patient fill when the prescribed amount of fresh PD fluid has been delivered.

During the patient dwell cycle 400b of FIG. 6B, disposable heating/mixing container or bag 104 may sit empty or close to empty as the final dialysis solution 102 dwells within the patient's peritoneal cavity. In another example, with a disposable heating/mixing container or bag 104 sized for multiple patient cycles, the disposable heating/mixing container or bag 104 may still contain enough final dialysis solution 102 for another therapy cycle. For example, as discussed above, the heater/mixing container 104 may include an upper chamber that serves as a drain chamber (e.g., drain chamber 105b of FIG. 1B) that receives waste fluid while another chamber (e.g., chamber 105a) of the heating/mixing container 104 holds the final dialysis solution 102 that is provided to the patient. Additionally, the heating/mixing container 104 may be part of a disposable set that includes a separate drain bag (see drain bag 615 of FIG. 7B).

Also, during the patient dwell cycle 400b, if there is fluid to be drained (e.g., drain fluid) in the drain bag 105b, the drain fluid may be moved from the drain bag 105b to the external drain 398. For example, a bag drain sequence (400d) may be performed during the patient dwell cycle 400b depending on the respective patient dwell cycle 400b of the entire treatment sequence. An example treatment sequence may be such that after patient dwell cycle 400b, a patient drain cycle 400c (drain bag fill) follows, followed by a patient fill cycle 400a, followed by another patient dwell cycle 400b (and corresponding bag drain 400d), and followed by another patient drain cycle (drain bag fill) 400c, etc.

During the patient drain cycle (bag fill) 400c of FIG. 6C, the used dialysis solution sitting in the patient's peritoneal cavity is pumped back into the empty drain chamber 105b of heater/mixing container 104 for weighing by weigh scale 290. Drain chamber 105b is sized to hold one drain volume's worth of drain fluid. After measuring the weight of drain fluid, the drain fluid is then pumped to a drain 398 (e.g., house drain) during the next patient dwell. Drain chamber 105b or drain bag 615 (see configuration illustrated in FIG. 4A and FIG. 7B) may have a 3 liter capacity while the primary chamber 105a of heater/mixing container 104 may have a 15 liter capacity. In another example, the primary chamber 105a may have a smaller capacity of approximately 6 liters for smaller patients or in scenarios where the system handles several bags or containers of fluid. Fluid valves 392a, 392c, 392d and 392f are closed. Additionally, fluid valves 392b and 392e are opened. For example, used dialysis fluid from the patient is passed back through the patient line 310 and open control valve 392e, through the pumping line segment 340 associated with the peristaltic pump 240, and through a waste line segment 344 and open control valve 392b to drain chamber 105b of heater/mixing container 104 or to a separate drain bag 615 (see configuration illustrated in FIG. 4A (105b) and FIG. 7B (615)). Control unit 296 is configured to end a patient drain either by removing a prescribed amount of effluent from the patient as weighed by weigh scale 290 or by sensing a characteristic negative pressure change (e.g., a sudden drop) via pressure sensor 280. In either instance, the amount of effluent drain from the patient is measured and recorded at control unit 296.

During the bag drain sequence 400d of FIG. 6D (e.g., performed during a patient dwell cycle 400b), the used dialysis fluid sitting in drain chamber 105b of heater/mixing container 104 or drain bag 615 (see configuration illustrated in FIG. 4A and FIG. 7B) is pumped to the drain 398. Fluid valves 392a, 392c, 392d and 392e are closed. Additionally, fluid valves 392b and 392f are opened. For example, used dialysis fluid from drain chamber 105b of heater/mixing container 104 or drain bag 615 (see configuration illustrated in FIG. 4A and FIG. 7B) is passed back through the waste line segment 344 and open control valve 392b, through the pumping line segment 340 associated with the peristaltic pump 240, and through a drain line 316 and open control valve 392f to the drain 398.

### Methods

In an embodiment, to begin treatment, a patient loads the disposable set 600 into the cycler 210 and in a random or designated order (i) places the heater/mixing container 104 or a heater/mixing bag (not pictured) onto cycler 210, (ii) connects upstream water line segment 630 to a water outlet connector of the water purifier 220, and (iii) connects drain line 616 to a drain connector of water purifier 220. Once fresh dialysis fluid is prepared and verified, the patient line 610 may be primed with fresh dialysis fluid, after which the patient may connect patient line connector 612 to a transfer set for treatment.

Alternatively, the disposable set 600 and the heater/mixing container 104 may be packaged together as a single disposable to minimize the amount of handling and connections required thereby advantageously minimizing the risk of introducing bacteria into the system when connecting the disposables together.

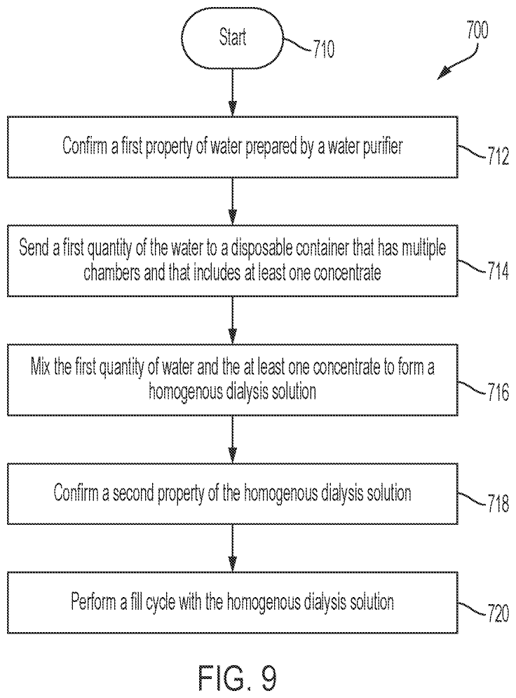

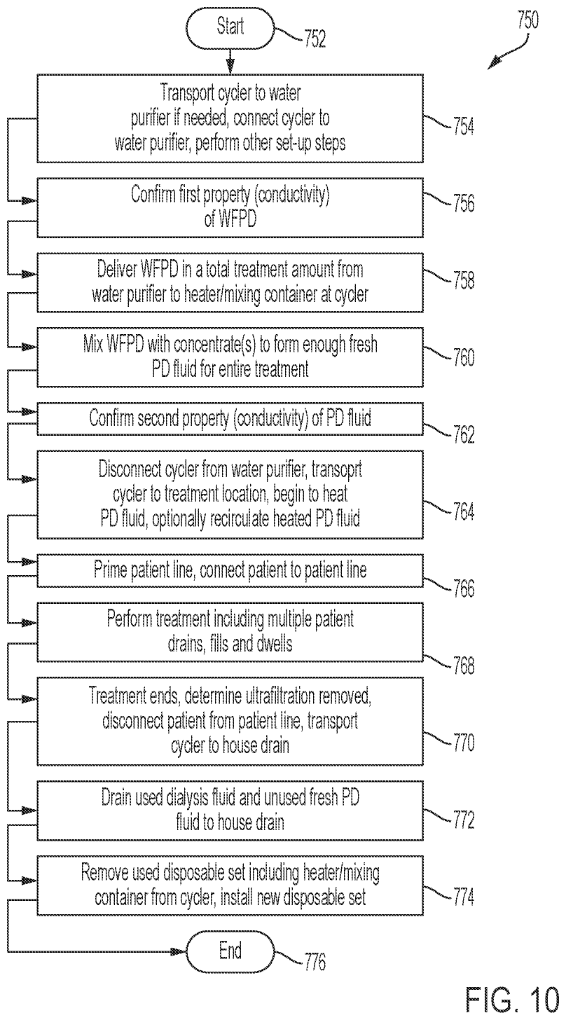

Referring now to FIG. 9, one embodiment for mixing dialysis solution and filling (or draining) the patient is illustrated by method 700. At oval 710, method 700 begins. At block 712, a first property of water purified by the water purifier 220 is confirmed. For example, control unit 286, 296 or separate device 235 may confirm a conductivity reading of the WFPD produced by the water purifier. The conductivity reading may be obtained from a conductivity cell 270 associated with the water purifier 220. When WFPD is at conductivity sensor 270, one or more conductivity readings may be obtained from conductivity sensor 270 for the WFPD. The conductivity readings may be compared to a look-up table to determine if the conductivity sensor reading is good or not. Then, at block 714, a first quantity of WFPD is sent to a heater/mixing container 104. As discussed above, the disposable heating/mixing container 104 may include multiple chambers, some of which may include a respective concentrate(s) 106.

Prior to confirming conductivity or another property of the WFPD or sending that water to the disposable heating/mixing container 104, a patient or other user may perform setup for system 200a, 200b as discussed above, including (i) turning the cycler 210 on, (ii) placing heater/mixing bag (not pictured) or heater/mixing container 104 onto cycler 210, (iii) connecting upstream water line segment 630 to water purifier 220, (iv) optionally connecting drain line 616 to water purifier 220, (v) connecting downstream line segment 632 to the heater/mixing container 104. Additionally, a control unit 296 of the cycler 210 may turn water purifier 220 on automatically, sync wirelessly with the water purifier's control unit 286, and tell the water purifier or its associated control unit 286 to prepare WFPD, e.g., specifying volume and temperature. Then, the water purifier 220 may pump a predetermined volume (e.g., 2 to 3 liters or more) of purified water at a predetermined temperature (e.g., 20° C. to 37° C., but preferably as close to a temperature near or at 37° C. such that the need for heating is minimized) through sterile sterilizing grade filters 260 to the heater/mixing container 104.

At block 716, a first quantity of WFPD may be mixed with the concentrate(s) 106 in the heater/mixing container 104 to form a homogenous dialysis solution. For example, after the proportioning phase, the contents of the disposable heating/mixing container 104 may be mixed according to the recirculation phase described herein. In an example, when mixing begins, the cycler 210 may be caused to open and close the appropriate fluid valves and operate the peristaltic pump 240 allowing fluid to pass through outlet 114 of heating/mixing container 104 and through mixing line segment 648 before returning through inlet 112 of heating/mixing container 104.

The cycler 210 is caused to (i) turn on the fluid heater to heat the mixture of WFPD and concentrate(s) 106 within the heater/mixing container 104 and (ii) perform a “recirculation” sequence. To perform the recirculation sequence or recirculation phase 300b, the concentrate(s) and water may be repeatedly pulled from outlet 114 of the heater/mixing container 104 and pushed back through inlet 112 of heater/mixing container 104. In an example, peristaltic pump 240 may be caused to (i) pull the mixture of WFPD and concentrate(s) 106 from heater/mixing container 104 into downstream line segment 332, 632, through an upstream portion of pumping line segment 340, 640 and (ii) push the mixture of WFPD and concentrate(s) 106 from a downstream portion of the pumping line segment 340, 640 and back to the disposable heating/mixing container inlet 112 through mixing line segment 348, 648.

Then, at block 718, a second property of the homogenous dialysis solution may be confirmed. Different PD dialysis fluids are typically differentiated by dextrose (glucose) levels. For example, there is a 4.25% dextrose monohydrate (or glucose monohydrate) PD dialysis fluid=3.86% anhydrous dextrose (or anhydrous glucose) PD dialysis fluid. 4.25% dextrose dialysis fluid may, depending on its chemical formulation, have a corresponding and repeatable conductivity measurement of 11.64 milli-siemens per centimeter (“mS/cm”). The other two common dialysis fluid types (1.5% dextrose and 2.5% dextrose) produce different corresponding and repeatable conductivity measurements. Control unit 296 can therefore verify if the dialysis solution 102 has been mixed properly by comparing its measured conductivity to an expected conductivity stored in a look-up table.

Next, at block 720, a fill cycle (or alternatively a dwell cycle or drain cycle depending on the stage of the therapy) is performed. For example, if the measured dialysis solution 102 is within the range of the setpoint conductivity, method 700 proceeds with treatment. For example, the cycler 210 or its associated control unit 296 may determine if the upcoming fill cycle 400a for the patient is a first fill cycle for the current treatment. Additionally, the cycler 210 or its associated control unit 296 may determine if the patient is already full with used dialysis fluid. If so, or if the upcoming fill cycle 400a is not the first fill cycle 400a, the method 700 may perform a drain cycle 400c for the patient. The drain cycle 400c may involve pumping the used dialysis fluid through the water generating device, such as water purifier 220. However, pumping the used dialysis fluid through the water purifier 220 may require a flow meter or other volumetric sensing equipment to determine the volume drained. If a flow meter or other volumetric sensing equipment is not present in the water purifier 220, the used dialysis fluid may first be weighed by weigh scale 290 before being sent to the water purifier 220 to drain. In an example, the heater/mixing container 104 is sized to allow one drain cycle 400 from the patient such that the entire quantity of the drain compartment or drain chamber 105b of heater/mixing container 104 is pumped to a drain between each APD cycle. If the patient does not have used dialysis fluid to initially drain, or when the drain cycle 400c is completed, method 700 may perform a fill cycle 400a for the patient. For example, the cycler 210 may be caused to open and close the appropriate fluid valves allowing fluid to pass from heating/mixing container 104 to the patient for a fill cycle. Additionally, the patient is ready for a patient drain cycle, the cycler 210 may be caused to open and close the appropriate fluid valves and operate the peristaltic pump 240 allowing used dialysis fluid to pass from the patient to the drain chamber 105b of heating/mixing container 104 or separate drain bag 615 before being passed through drain line 616 to the drain.

Additionally, method 700 may perform a patient dwell cycle 400b. During the dwell cycle 400b, the cycler 210 is caused to close control valve 392e to patient line 610 (during the dwell cycle 400b, each of the fluid valves 292, 392 may be closed). The therapeutic effect of the newly mixed fresh dialysis solution 102 takes place during the dwell cycle 400b, e.g. where waste products and toxins are removed by diffusion and convection from the blood of the patient, through patient's peritoneal membrane, into the dialysis fluid. Excess fluid from the patient is also removed into the dialysis fluid as ultrafiltration (“UF”), typically seven percent of the fill volume, so roughly 140 milliliters for a 2 liter fill volume). The dwell cycle 400b may last one to two hours, for example.

At oval 722, method 700 ends.

Referring now to FIG. 10, another embodiment for mixing dialysis solution and performing a peritoneal dialysis treatment on the patient is illustrated by method 750. At oval 752, method 750 begins. In method 750, water purifier 220 is located close to a source of tap water in the patient's home, e.g., in a bathroom that is part of or next to the patient's bedroom (hereafter the “water/drain room”). The location of water purifier 220 being close to a source of tap water greatly reduces the length and complexity of a hose, pipe, and/or tubing connection between the source of tap water and water purifier 220. In addition to being less costly and less intrusive for the patient, the reduced tap water and water purifier 220 connection also reduces the risk of microbial growth and water leakage. The hose, pipe, and/or tubing connection between the source of tap water and water purifier 220 may be permanent or semi-permanent.

At block 754, the patient or caregiver, if needed, transports, e.g., wheels cycler 210 of system 200a, 200b (or any cycler of any system described herein) into the water/drain room where water purifier 220 resides. Due to PD being a daily and continuous treatment, it should be appreciated that cycler 210 is likely already located in the tap water/drain room due to the need drain used dialysis fluid from a previous treatment, which is typically performed at the end of the previous treatment. In either case, the patient or caregiver fluidly connects cycler 210 to the water purifier, e.g., via water line 250. The connection 250 between cycler 210 and water purifier 220 may also be short, reducing cost, complexity, microbial growth and water leakage. At block 754, the patient or caregiver performs any other needed procedures in the tap water/drain room for setting up system 200a, 200b, including (i) turning the cycler 210 and water purifier 220 on, and (ii) removing a spent disposable set and placing a new disposable set including a heater/mixing container 104 onto cycler 210, if not done at the end of the last treatment. Control unit 296 of cycler 210 may alternatively turn water purifier 220 on automatically, sync wirelessly with the water purifier's control unit 286, and tell the water purifier or its associated control unit 286 to prepare WFPD, e.g., specifying a volume to prepare. With method 750, the amount of WFPD and resulting dialysis fluid prepared is for an entire treatment, e.g., six liters or more.

At block 756, a first property of water purified by the water purifier 220 is confirmed. For example, control unit 286, 296 or separate device 235 may confirm a conductivity reading of the WFPD produced by water purifier 220. The conductivity reading may be obtained from a conductivity cell 270 associated with the water purifier 220. The conductivity reading may be compared to a look-up table stored in control unit 286, 296 or separate device 235 to determine whether or not the conductivity sensor reading indicates that the WFPD is proper for treatment.

At block 758, a total treatment quantity of WFPD is pumped from water purifier 220 to heater/mixing container 104 at cycler 210, e.g., via water purifier pump 225 or cycler pump 240. As discussed above, the disposable heating/mixing container 104 may include multiple chambers, one or more of which (e.g., fresh dialysis fluid chamber 105a) may include a respective concentrate(s) 106, e.g., enough to make a total treatment's worth of dialysis fluid. In one embodiment, WFPD delivery and dialysis fluid preparation are performed long before treatment, e.g., during the day while the patient is working or performing a daily routine. The WFPD accordingly does not need to be heated before delivery from water purifier 220 to heater/mixing container 104.

At block 760, a first quantity of WFPD may be mixed with the concentrate(s) 106 in the heater/mixing container 104 to form enough homogenous dialysis solution for an entire treatment. For example, after the proportioning phase at block 758, the concentrate(s) 106 preloaded into disposable heating/mixing container 104 may be mixed according to any recirculation phase described herein. In an example, when mixing begins, the cycler 210 may be caused to open and close the appropriate fluid valves and operate peristaltic pump 240, causing fluid to pass through outlet 114 of heating/mixing container 104 and through mixing line segment 648 (FIG. 7B) before returning through inlet 112 of heating/mixing container 104.

At block 762, a second property of the homogenous dialysis solution may be confirmed, e.g., via pumping the solution past conductivity sensor 270 of cycler 210, which outputs to control unit 296. Different PD dialysis fluids are typically differentiated by dextrose or glucose levels and produce different corresponding and repeatable conductivity measurements as has been discussed herein. The control unit 296 can therefore verify if the dialysis solution 102 has been mixed properly by comparing its measured conductivity to an expected conductivity stored, e.g., in a look-up table at control unit 296. Thus, by the end of block 762, an entire treatment's worth of dialysis fluid has been prepared and confirmed and which resides at cycler 210.

At block 764 it is nearing time for treatment. The patient or caregiver disconnects cycler 210 from water purifier 220 and transports, e.g., wheels the cycler from the water/drain room to the location of treatment, e.g., next to the patient's bed at nighttime before it is time to sleep. Cycler 210 is then caused to (i) turn on fluid heater 294 to heat properly mixed dialysis solution 102 within the heater/mixing container 104 and (ii) may optionally perform a “recirculation” sequence described in connection with method 700 to promote even heating. The entire treatment's worth of dialysis fluid is heated to a treatment temperature, e.g., 37° C. or body temperature, via heater 294 under the control of control unit 296 receiving feedback output from one or more pressure sensor. Advantageously, heating of the dialysis fluid within the heater/mixing container 104 may be performed while the patient is away from cycler 210, relaxing or performing other tasks (e.g., fluid heating may be started automatically at a time prior to an expected treatment start time so that the dialysis fluid is already heated at the start of treatment).

At block 766, the patient connects (in a sterile manner) the patient line extending from cycler 210 to the patient's transfer set. The connection is made aseptically in a manner known in the art. The patient line may be primed with fresh, heated dialysis fluid prior to or after the connection of the patient line to the patient's transfer set.

At block 768, cycler 210 performs treatment on the patient using the properly mixed and heated dialysis solution. As discussed herein, the first sequence of the treatment may be a patient drain cycle 400c described in connection with FIG. 6C, which is then followed by a patient fill cycle 400a described in connection with FIG. 6A and a dwell cycle 400b described in connection with FIG. 6B. The above cycles are repeated a number of times according to the patient's treatment prescription carried out by control unit 296 of cycler 210. Alternatively, the first sequence of the treatment may be a patient fill cycle 400a, which is then followed by a dwell cycle 400b and a patient fill cycle 400. The above alternative cycles are likewise repeated a number of times according to the patient's treatment prescription.

Block 770 occurs in one example in the morning at the end of treatment. Fresh dialysis fluid chamber 105a of container 104 is now empty or almost empty. Drain chamber 105b of container 104 is now full or almost full from being filled with multiple drains' worth of patient effluent. Control unit 296 receiving an output from weigh scale 290 is able to subtract (i) a total initial weight of fresh dialysis fluid prepared within container 104 prior to the first drain or fill of the treatment the night before from (ii) the weight of the total patient effluent (and any residual fresh fluid) located in container 104 the morning after treatment is completed, to determine the amount of ultrafiltration removed from the patient over the course of treatment. The patient disconnects from the patient line extending from cycler 210 and transports, e.g., wheels, the cycler into the tap water/drain room containing water purifier 220.

At block 772, the user places the drain line extending from cycler 210 into a toilet, bathtub or other receptacle located within the tap water/drain room. The user then presses a “drain effluent bag” button provided by a touch screen associated with display device 230. Control unit 296 then causes cycler 210 to drain the effluent fluid (and perhaps any remaining fresh dialysis fluid) from container 104 to the house drain, e.g., according to bag drain procedure 400d described in connection with FIG. 6D.

At block 774, after the drain from container 104 to the house drain is completed, display device 230 audibly, visually or audiovisually prompts the patient or user to discard the disposable set including container 104 and to load a new disposable set into cycler 210. After loading a new, e.g., presterilized, disposable set into cycler 210, method 750 is able to be repeated.

At oval 776, method 750 ends.

### Glucose (Dextrose)

Glucose (Dextrose) is a highly reactive substance and is easily degraded into Glucose Degradation Products (“GDPs”), which are toxic and can give rise to early or advanced glycation end products. The formation of GDP is dependent on pH, temperature and time. GDPs are generated in PD fluids when glucose is heated (e.g., as under heat sterilization) as well as subsequently during storage of the sterilized fluids. For example, some glucose-containing medical solutions are subject to glucose degradation or aggregation when sterilized using conventional moist heat sterilization techniques.

It is known that carbohydrates such as glucose can degrade during conventional heat sterilization procedures such as autoclaving to form toxic or otherwise undesirable glucose degradation products within the sterilized solution. The degradation of glucose in medical solutions results in the formation of GDPs that may be cytotoxic, may induce pro-inflammatory activation signals, and may promote formation of advanced glycation end products (“AGEs”) that some studies have suggested cause vascular damage to peritoneal dialysis patients.

Non-limiting examples GDPs include 3-deoxyglucosone (3-DG), 5-hydroxymethylfurfural (5-HMF), glyoxal, methylglyoxal (MeGly), formaldehyde, acetaldehyde, 3,4-dideoxyglucosone-3-ene (3,4-DGE), furfural, and many other substances that have not yet been identified chemically. It has been suggested that over time the damage caused by GDPs and AGEs may severely impair the filtering capability of the peritoneal membrane, which may ultimately force a PD patient to switch to a less convenient dialysis therapy such as hemodialysis.

By creating dialysis solution 102 on-site from disposable bags 104 that only include a concentrate(s) 106 instead of being shipped with finished dialysis solution, the formation of GDPs from sterilization and storage are advantageously reduced. Additionally, using the concentrates of the present disclosure, e.g., having 50% or higher glucose concentrations, tends to create less GDPs.

### Cycler/Water Purifier/Monitor Communication

The cycler 210 may pair or sync with water purifier 220 via wired or wireless communication between control units 286 and 296. Once wirelessly paired, cycler 210 may order WFPD as needed from water purifier 220. For example, cycler 210 may specify a quantity and temperature for the WFPD. Additionally, cycler 210 may specify a maximum WFPD supply pressure. If needed, cycler 210 may also tell water purifier 220 to abort the previously ordered delivery, e.g., if cycler 210 has experienced an alarm that is currently being addressed or if the patient has ended treatment for whatever reason. Similarly, separate device 235 may pair and sync with one or both of the cycler 210 and water purifier 220 to obtain, monitor and analyze sensor data obtained from either the cycler 210 or water purifier 220.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1: A peritoneal dialysis system comprising:
a water purifier;
a disposable set including
a water line in fluid communication with the water purifier,
a drain line for draining from the disposable set, and
a disposable container including at least one chamber, the disposable container including at least one concentrate in one of the at least one chamber, and the disposable container positioned and arranged to hold a dialysis fluid prepared by mixing water from the water purifier and the at least one concentrate; and
a control unit in communication with a sensor, for detecting a conductivity of the water from the water purifier and a conductivity of the dialysis fluid.

2: The peritoneal dialysis system of claim 1, wherein the sensor is a conductivity sensor.

3. (canceled)

4: The peritoneal dialysis system of claim 1, wherein the sensor is housed within the water purifier.

5: The peritoneal dialysis system of claim 1, wherein the sensor is at least partially housed within the disposable set.

6: The peritoneal dialysis system of claim 1, further comprising at least one of a weigh scale for weighing the disposable container or a heater for heating the dialysis fluid.

7: The peritoneal dialysis system of claim 1, further comprising a cycler configured to (i) mix water from the water purifier and the at least one concentrate to form a dialysis fluid and (ii) deliver the dialysis fluid to a patient.

8: The peritoneal dialysis system of claim 7, wherein the cycler includes a heating pan configured to hold the disposable container and to heat an area within the disposable container.

9: The peritoneal dialysis system of claim 7, wherein the control unit is part of the cycler.

10: The peritoneal dialysis system of claim 1, wherein the control unit stores a look-up table with at least one setpoint value for at least one of the conductivity of the water from the water purifier or the conductivity of the dialysis fluid.

11: The peritoneal dialysis system of claim 1, wherein the control unit is programmed to compare a sensed value for at least one of the conductivity of the water from the water purifier or the conductivity of the dialysis fluid sensed by the to a setpoint value.

12: The peritoneal dialysis system of claim 1, wherein one of the at least one chamber of the disposable set is adapted for receiving used dialysis fluid from a patient drain.

13: The peritoneal dialysis system of claim 1, wherein the disposable set includes a pumping tube for operation with a peristaltic pump.

14: A peritoneal dialysis system comprising:
a disposable set including
a water line for fluid communication with a water source,
a disposable container including at least one concentrate, the disposable container positioned and arranged to hold a dialysis fluid prepared by mixing purified water from the water source and the at least one concentrate to form the dialysis fluid,
a pumping line segment in fluid communication with the disposable container,
a patient line downstream of the pumping line, and
a mixing line segment in fluid communication with the disposable container and the pumping line; and
a cycler including a pump positioned along the pumping line, the cycler configured to perform at least one of (i) a recirculation phase for mixing purified water with the at least one concentrate using the mixing line, (ii) a patient fill for delivering the dialysis fluid to a patient, (iii) a patient drain for removing used dialysis fluid from the patient, and (iv) moving drain fluid from the disposable container to a drain.

15: The peritoneal dialysis system of claim 14, further comprising at least one sensor for confirming a property of at least one of the purified water or the dialysis fluid.

16: The peritoneal dialysis system of claim 14, wherein the pump is a peristaltic pump operating with the pumping line segment.

17: The peritoneal dialysis system of claim 14, wherein the cycler includes a weigh scale configured to weigh dialysis fluid delivered from the disposable container during the patient fill and the used dialysis fluid delivered to the disposable container during the patient drain.

18: The peritoneal dialysis system of claim 14, wherein the cycler includes a heater configured to heat purified water and the at least one concentrate mixed during the recirculation phase.

19: The peritoneal dialysis system of claim 14, wherein the cycler includes a pressure sensor positioned and arranged to measure a patient fill pressure of dialysis fluid delivered from the disposable container during the patient fill and a patient drain pressure of the used dialysis fluid delivered to the disposable container during the patient drain.

20: The peritoneal dialysis system of claim 14, wherein the cycler is configured such that when performing the recirculation phase of (i), the cycler causes:
the pump to pull a mixture of the purified water and the at least one concentrate from the outlet of the disposable container, through a downstream line segment, and into an upstream portion of the pumping line segment, and
the pump to push the mixture of water and the at least one concentrate to a downstream portion of the pumping line segment through the mixing line segment, and into the disposable container through the inlet to form the dialysis fluid.

21: The peritoneal dialysis system of claim 20, wherein the cycler is configured such that when performing the recirculation phase of (i), is the cycler causes the pump to push the mixture of purified water and the at least one concentrate to a sensing line segment positioned between the pumping line segment and the mixing line segment.

22: The peritoneal dialysis system of claim 14, wherein the cycler is configured such that when performing the patient fill of (ii), the cycler causes:
the pump to pull the dialysis fluid from the outlet of the disposable container, through a downstream line segment, and into an upstream portion of the pumping line segment, and
the pump to push the dialysis fluid to a downstream portion of the pumping line segment and through the patient line.

23: The peritoneal dialysis system of claim 22, wherein the cycler is configured such that when performing the patient fill of (ii), the cycler causes the pump to push the dialysis fluid to a sensing line segment positioned between the pumping line segment and the patient line.

24: The peritoneal dialysis system of claim 14, wherein the cycler is configured such that when performing the patient drain of (iii), the cycler causes:
the pump to pull the used dialysis fluid from a patient, through the patient line, and through a downstream portion of the pumping line segment, and
the pump to push the used dialysis fluid to an upstream portion of the pumping line segment, through a waste line segment to a drain compartment of the disposable container.

25: The peritoneal dialysis system of claim 24, wherein the cycler is configured such that when performing (iv) and moving drain fluid from the disposable container to a drain, the cycler causes the pump to pull the used dialysis fluid from the drain compartment of the disposable container and push the used dialysis fluid to a house drain or terminal drain container.

26-42. (canceled)
